SEQUENCE LISTING (60 sequences)

SEQ ID NO: 1 - DNA, 1835 bp, Homo sapiens
ttttacagttttccttttcttcagagtttattttgaattttcatttttggataaccaagcagctctttaagaagaatgcacagaagagtcattctggcacttttggatagtacataagattttcttttttttttttaaattttttttaatagtcacattcagctcgcttgctcaaaccagactcccacattgggtgagcaagatgagcccataggattccagagttaatacgtaaccgtatatacaaacagccaaaaaaccataatggtgccacagggatggagcagggaagggcatctctaacgtgtcctctagtctatcttcgctaaacagaacccacgttacacatgataactagagagcacactgtgttgaaacgaggatgctgaccccaaatggcacttggcagcatgcagtttaaagcaaaagagacatcctttaataactgtataaaatccaggcagttccattaaaggggttaagaaaaccaacaacaacaaaaagcgagggactgtctgttgtcactgtcaaaaaggcacttggagttaatgggaccaggattggaggactcttagctgatacagatttcagtacgatttcattaaaaggcttggatgttaagagaggacactcagcggttcctgaagggagacgctgagatggaccgctgagaagcggaacagatgaacacaaaggaatcaaatctttacaaccaaattgcatttaagcgacaacaaaaaaaggcaaaccccaaaacgcaacctaaccaaagcaaaatctaagcaaaatcagacaacgaagcagcgatgcatagctttcctttgagagaacgcataccttgagacgctacgtgccaacctaagttctcaacgacagcttcacagtaggattattgtgataaaaatgactcaagcgatgcaaaaagtttcatctgttcccagaatccgagggagaactgaggtgatcgttagagcatagcgacatcacgtgcggtttcttaatgtccctggtggcggatacgccgagtcctcggaaggacatctggacaccactttcagccacctccttgcaggggcgacatccgccaaagtcatcctttattccgagtaataactttaattcctttctaacatttacacggcaaacaggaatgcagtaaacgtccacgtccgtcccacggctgggctgccgttccgtttcctccacgaacgggtacgcgcttccatgagaaaggatatttggcaattttatattccacagtcaggtgggtctgcgatagctcatttaatgttaaacgccatcaggggcctctcctcccgtttctgccaggggcttttcttgtcttctccttggcgagctcgtgggcagatcttctctggtgggggctggctgctggctccgagggggcatccgcagtccgtctggtcgtctcctcctgcaggctgggcagctggccaccacttctccgactcgacccctccaacaagcatcgcagggcactgtcctcgggggtacagaccgtggtcccacattcgctaccactctgttccacgtcatccaggtacacgagctgcgtgtaggccgtgctgtctggggctcgaggctctttctgctggtgctcttggacgggcgggtagttctgctgcagagacaaagcatctccccttcccttccgggctgattttggttcattcatatctacgccagagtccaaactggcatcattacttccgttccttccagctctttggagaatcaatgtatgaatgtctaacctgaccgttggacctgccatccaaggagacgaaccacgcccgggggtgcggaagcggcct

SEQ ID NO: 2 - DNA, 581 bp, Homo sapiens
gttctagattgttttattcagtaattagctcttaagacccctggggcctgtgctacccagacactaacaacagtctctatccagttgctggttctgggtgacgtgatctccccatcatgatcaacttacttcctgtggcccattagggaagtggtgacctcgggagctatttgcctgttgagtgcacacacctggaaacatactgctctcattttttcatccacatcagtgagaaatgagtggcccgttagcaagatataactatgcaatcatgcaacaaagctgcctaataacatttcatttattacaggactaaaagttcattattgtttgtaaaggatgaattcataacctctgcagagttatagttcatacacagttgatttccatttataaaggcagaaagtccttgttttctctaaatgtcaagctttgactgaaaactcccgtttttccagtcactggagtgtgtgcgtatgaaagaaaatctttagcaattagatgggagagaagggaaatagtacttgaaatgtaggccctcacctccccatgacatcctccatgagcctcctgatgtagtg

SEQ ID NO: 3 - DNA, 516 bp, Homo sapiens
tagagatgttggttgatgacccccgggatctggagcagatgaatgaagagtctctggaagtcagcccagacatgtgcatctacatcacagaggacatgctcatgtcgcggaacctgaatggacactctgggttgattgtgaaagaaattgggtcttccacctcgagctcttcagaaacagttgttaagcttcgtggccagagtactgattctcttccacagactatatgtcggaaaccaaagacctccactgatcgacacagcttgagcctcgatgacatcagactttaccagaaagacttcctgcgcattgcaggtctgtgtcaggacactgctcagagttacacctttggatgtggccatgaactggatgaggaaggcctctattgcaacagttgcttggcccagcagtgcatcaacatccaagatgcttttccagtcaaaagaaccagcaaatacttttctctggatctcactcatgatgaagttccagagtttgttgtgtaaagtccgtctg

SEQ ID NO: 4 - DNA, 1099 bp, Homo sapiens
cccacaacacaggggccctgaaacacgccagcctctcctctgtggtcagcttggcccagtcctgctcactggatcacagcccattgtaggtggggcatggtggggatcagggcccctggcccacggggaggtagaagaagacctggtccgtgtaagggtctgagaaggtgccctgggtcgggggtgcgtcttggccttgccgtgccctcatcccccggctgaggcagcgacacagcaggtgcaccaactccagcaggttaagcaccagggagatgagtccaaccaccaacatgaagatgatgaagatggtcttctccgtggggcgagagacaaagcagtccacgaggtaggggcagggtgctcgctggcacacaaacacgggctccatggtccagccgtacaggcgccactggccatagaggaagcctgcctctagcacactcttgcagagcacactggcgacataggtgcccatcagtgctccgcggatgcgcaggcgaccatcttctgccaccgagatcttggccatctgacgctctacggccgccagcgcccgctccacctgtgggtccttggccggcagtgcccgcagctccccctccttctgccgcagccgctcttctcgccgagacaggtaaatgacatggcccaggtagaccagggtgggtgtgctgacgaagaggaactgcagcacccagtagcggatgtgggagatggggaaggcctggtcatagcagacgttggtgcagcctggctgggccgtgttacactcgaaatctgactgctcgtcaccccacactgactcgccggccaggcccaggatgaggatgcggaagatgaagagcaccgtcagccagatcttacccaccacggtcgagtgctcctggacctggtccagcaacttctccacgaagccccagtcacccatggctcccgggcctccgtcggcaaggagacagagcacgtcagtgtgtcagcatggcatccttctcgttcgcccagcaacaagcctgcagggaggtctgccacgcccgttctaccgcctgcctgccgggcggcccaggtggaggtggggacgatggccggagtgacgcccgcg

SEQ ID NO: 5 - DNA, 1015 bp, Homo sapiens
gaggatagggagcctggggtcaggagtgtgggagacacagcgagactctgtctccaaaaaaaaaagtgctttttgaaaatgttgaggttgaaatgatgggaaccaacattctttggatttagtggggagcataatagcaaacacccccttggttcgcacatgtacaggaatgggacccagttggggcacagccatggacttccccgccctggaatgtgtggtgcaaagtggggccagggcccagacccaagaggagagggtggtccgcagacaccccgggatgtcagcatcccccgacctgccttctggcggcacctcccgggtgctgtgttgagtcagcaggcatggggtgagagcctggtatatgctgggaacagggtgcaggggccaagcgttcctccttcagccttgacttgggccatgcaccccctctcccccaaacacaaacaagcacttctccagtatggtgccaggacaggtgtcccttcagtcctctggttatgacctcaagtcctacttgggccctgcagcccagcctgtgttgtaacctctgcgtcctcaagaccacacctggaagattcttcttccctttgaaggagaatcatcattgttgctttatcacttctaagacattttgtacggcacggacaagttaaacagaatgtgcttccctccctggggtctcacacgctcccacgagaatgccacaggggccgtgcactgggcaggcttctctgtagaaccccaggggcttcggcccagaccacagcgtcttgccctgagcctagagcagggagtcccgaacttctgcattcacagaccacctccacaattgttataaccaaaggcctcctgttctgttatttcacttaaatcaacatgctattttgttttcactcacttctgactttagcctcgtgctgagccgtgtatccatgcagtcatgttcacgtgctagttacgtttttcttcttacacatgaaaataaatgcataagtgttagaagaaaaaaaaa

SEQ ID NO: 6 - DNA, 2313 bp, Homo sapiens
ccagagcaggcctggtggtgagcagggacggtgcaccggacggcgggatcgagcaaatgggtctggccatggagcacggagggtcctacgctcgggcggggggcagctctcggggctgctggtattacctgcgctacttcttcctcttcgtctccctcatccaattcctcatcatcctggggctcgtgctcttcatggtctatggcaacgtgcacgtgagcacagagtccaacctgcaggccaccgagcgccgagccgagggcctatacagtcagctcctagggctcacggcctcccagtccaacttgaccaaggagctcaacttcaccacccgcgccaaggatgccatcatgcagatgtggctgaatgctcgccgcgacctggaccgcatcaatgccagcttccgccagtgccagggtgaccgggtcatctacacgaacaatcagaggtacatggctgccatcatcttgagtgagaagcaatgcagagatcaattcaaggacatgaacaagagctgcgatgccttgctcttcatgctgaatcagaaggtgaagacgctggaggtggagatagccaaggagaagaccatttgcactaaggataaggaaagcgtgctgctgaacaaacgcgtggcggaggaacagctggttgaatgcgtgaaaacccgggagctgcagcaccaagagcgccactggccaaggagcaactgcaaaaggtgcaagccctctgcctgcccctggacaaggacaagtttgagatggaccttcgtaacctgtggagggactccattatcccacgcagcctggacaacctgggttacaacctctaccatcccctgggctcggaattggcctccatccgcagagcctgcgaccacatgcccagcctcatgagctccaaggtggaggagctggcccggagcctccgggcggatatcgaacgcgtggcccgcgagaactcagacctccaacgccagaagctggaagcccagcagggcctgcgggccagtcaggaggcgaaacagaaggtggagaaggaggctcaggcccgggaggccaagctccaagctgaatgctcccggcagacccagctagcgctggaggagaaggcggtgctgcggaaggaacgagacaacctggccaaggagctggaagagaagaagagggaggcggagcagctcaggatggagctggccatcagaaactcagccctggacacctgcatcaagaccaagtcgcagccgatgatgccagtgtcaaggcccatgggccctgtccccaacccccagcccatcgacccagctagcctggaggagttcaagaggaagatcctggagtcccagaggccccctgcaggcatccctgtagccccatccagtggctgaggaggctccaggcctgaggaccaagggatggcccgactcggcggtttgcggaggatgcagggatatgctcacagcgcccgacacaaccccctcccgccgcccccaaccacccagggccaccatcagacaactccctgcatgcaaacccctagtaccctctcacacccgcacccgcgcctcacgatccctcacccagagcacacggccgcggagatgacgtcacgcaagcaacggcgctgacgtcacatatcaccgtggtgatggcgtcacgtggccatgtagacgtcacgaagagatatagcgatggcgtcgtgcagatgcagcacgtcgcacacagacatggggaacttggcatgacgtcacaccgagatgcagcaacgacgtcacgggccatgtcgacgtcacacatattaatgtcacacagacgcggcgatggcatcacacagacggtgatgatgtcacacacagacacagtgacaacacacaccatgacaacgacacctatagatatggcaccaacatcacatgcacgcatgccctttcacacacactttctacccaattctcacctagtgtcacgttcccccgaccctggcacacgggccaaggtacccacaggatcccatcccctcccgcacagccctgggccccagcacctcccctcctccagcttcctggcctcccagccacttcctcacccccagtgcctggacccggaggtgagaacaggaagccattcacctccgctccttgagcgtgagtgtttccaggaccccctcggggccctgagccgggggtgagggtcacctgttgtcgggaggggagccactccttctcccccaactcccagccctgcctgtggcccgttgaaatgttggtggcacttaataaatattagtaaatccttaaaaaaaaaaaaaaaaaaaaaa

SEQ ID NO: 7 - DNA, 389 bp, Homo sapiens
gccaaaaagatggcttcaaaagtaagaatgaaacatttgatccattcagctttaggctatgccactggattcatgtctagaaaagataggataatttctgtaaagaaatgaagaccttgctattctaaaatcagatccttacagatccagatttcaggaaacaaatacataggggactaactttccttgttcagattagtttttctcctttgcacccagctatataatatgaggaagtattgactttttaaaagtgttttagttttccatttctttgatatgaaaagtaatatttcgggagaaccctgagctattaataatctatgtggctagtgcgtatatattggtctgaatttgttctccttttgtggtgtccagtgggtaacatc

SEQ ID NO: 8 - DNA, 157 bp, Homo sapiens
tgctttaaacagctgtgtcaaaaactgacatcagagagtaaattgaatttggttttgtaggaagcaggaagcaagcccactcaaacgtgaaatttggcatgagggatccagtaactttctcctcaatctgtgaactatatgtgagtttgatattttg

SEQ ID NO: 9 - DNA, 561 bp, Homo sapiens
aatagtcaaaacataaacaaaagctaattaactggcactgttgtcacctgagactaagtggatgttgttggctgacatacaggctcagccagcagagaaagaattctgaattccccttgctgaactgaactattctgttacatatggttgacaaatctgtgtgttatttcttttctacctaccatatttaaatttatgagtatcaaccgaggacatagtcaaaccttcgatgatgaacattcctgattttttgcctgattaatctctgttgagctctacttgtggtcattcaagattttatgatgttgaaaggaaaagtgaatatgacctttaaaaattgtattttgggtgatgatagtctcaccactataaaactgtcaattattgcctaatgttaaagatatccatcattgtgattaattaaacctataatgagtattcttaatggagaattcttaatggatggattatcccctgatcttttctttaaaatttctctgcacacacaggacttctcattttccaataaatgggtgtactctgccccaatttctaggaaaa

SEQ ID NO: 10 - DNA, 1508 bp, Homo sapiens
cacaaacacgagagactccacggtctgcctgagcaccgccagcctcctaggctccagcactcgcaggtccattcttctgcacgagcctctctgtccagatccataagcacggtcagctcagggtcgcggagcagtacgaggacaagtaccagcagcagctcctctgaacagagactgctaggatcatccttctcctccgggcctgttgctgatggcataatccgggtgcaacccaaatctgagctcaagccaggtgagcttaagccactgagcaaggaagatttgggcctgcacgcctacaggtgtgaggactgtggcaagtgcaaatgtaaggagtgcacctacccaaggcctctgccatcagactggatctgcgacaagcagtgcctttgctcggcccagaacgtgattgactatgggacttgtgtatgctgtgtgaaaggtctcttctatcactgttctaatgatgatgaggacaactgtgctgacaacccatgttcttgcagccagtctcactgttgtacacgatggtcagccatgggtgtcatgtccctctttttgccttgtttatggtgttaccttccagccaagggttgccttaaattgtgccaggggtgttatgaccgggttaacaggcctggttgccgctgtaaaaactcaaacacagtttgctgcaaagttcccactgtcccccctaggaactttgaaaaaccaacatagcatcattaatcaggaatattacagtaatgaggattttttctttctttttttaatacacatatgcaaccaactaaacagttataatcttggcactgttaatagaaagttgggatagtctttgctgtttgcggtgaaatgctttttgtccatgtgccgttttaactgatatgcttgttagaactcagctaatggagctcaaagtatgagatacagaacttggtgacccatgtattgcataagctaaagcaacacagacactcctaggcaaagtttttgtttgtgaatagtacttgcaaaacttgtaaattagcagatgacttttttccattgttttctccagagagaatgtgctatatttttgtatatacaataatatttgcaactgtgaaaaacaagtggtgccatactacatggcacagacacaaaatattatactaatatgttgtacattcggaagaatgtgaatcaatcagtatgtttttagattgtattttgccttacagaaagcctttattgtaagactctgatttccctttggacttcatgtatattgtacagttacagtaaaattcaacctttattttctaattttttcaacatattgtttagtgtaaagaatatttatttgaagttttattattttataaaaaagaatatttattttaagaggcatcttacaaattttgccccttttatgaggatgtgatagttgctgcaaatgaggggttacagatgcatatgtccaatataaaatagaaaatatattaacgtttgaaattaaaaaaaaa

SEQ ID NO: 11 - DNA, 389 bp, Homo sapiens
gggcaggtgatcagggcacacatttcccgtccattgagacagtagcattcccggcacccatcgtgccagctctcctcatttttatgatgatgaccatccacggtgagacaagtgcccgacaggatgggtggcccagctgaagcacaggccgctctgcacttgcagataagacagccgtgactgtcctgctggaaacccaaggggcagatcttactgcatgagagctctggacatttcttacagcgacagatgtcacagccgtgcttattcttcagcaatccaagtggacaatacttgtcacagattatgggtctgcacttcttgggccttgggcggcactcacagatctcacagttttggacctcggccgcgaccacgctgggtaccga

SEQ ID NO: 12 - DNA, 981 bp, Homo sapiens
ttttttttttttggattgcaaaaatttattaaaattggagacactgttttaatcttcttgtgccatgagactccatcaggcagtctacaaagaccactgggaggctgaggatcacttgagcccagaagtttgaggctgtagtaagcttcaaaggccactgcactctagcttgggtgaggcaagaccctttcaagcagtaagctgcatgcttgcttgttgtggtcattaaaaaccctagtttaggataacaacatattaatcagggcaaaatacaaatgtgtgatgcttgttagtagagtaacctcagaatcaaaatggaacggttttacagtgatatcattatatttcatttggcagaatcattacatcattggttacactgaaaatcatcacatgtaccaaaagctgactcacctagtttaggataacaggtctgcctgtttgaagatgaaaaataatacccatttaaaatttgccctactcaatttccttctcagtcacattttaacttttaaacagctaatcactcccatctacagattaaggtgtatatgccaccaaaaccttttgccaccttaaaaatttccttcaaagtttaaactaatgcctgcatttcttcaatcatgaattctgagtcctttgcttctttaaaacttgctccacacagtgtagtcaagccgactctccatacccaagcaagtcatccatggataaaaacgttaccaggagcagaaccattaagctggtccaggcaagttggactccaccatttcaacttccagctttctgtctaatgcctgtgtgccaatggcttgagttaggcttgctctttaggacttcagtagctattctcatccttccttggggacacaactgtccataaggtgctatccagagccacactgcatctgcacccagcaccatacctcacaggagtcgactcccacgagccgcctgtatataagagttcttttgatgacg

SEQ ID NO: 13 - DNA, 401 bp, Homo sapiens
ataactacagcttcagcagacaactaaagagactgcattaaggtgatttctctggctataaagagagcccggccgcagagcatgtgactgctgggacctctgggataggcaacactgccctctctcccccagagcgaccccccgggcaggtcggggcccaaggaatgacccagcaactgctccctacccagcacactctctttactgccacctgcaattatgctgtgaagatgactgggtgtggtcatcacgattcagagaaatcaagatctatgaccattttaggcaaagagagaaacttggagaattgctgaggactactgaaccttgttttgcttttttaaaaaatactaaatcctcacttcagcatatttagttgtcattaaaattaagctgatatt

SEQ ID NO: 14 - DNA, 1002 bp, Homo sapiens
gacaatataaaaagtggaaacaagcataaattgcagacataaaataatcttctggtagaaacagttgtggagaacaggttgagtagagcaacaacaacaaaagcttatgcagtcaccttctttgaaaatgttaaatacaagtcctattctctttgtccagctgggtttagctagaggtagccaattacttctcttaaggtccatggcattcgccaggattctataaaagccaagttaactgaagtaaatatctggggcccatcgcacccccactaagtactttgtcaccatgttgtatcttaaaagtcatttttcactgtttgactcagaatttgggacttcagagtcaaacttcattgcttactccaaacccagtttaattccccacttttttaagtaggcttagctttgagtgatttttggctataaccgaaatgtaaatccaccttcaaacaacaaagtttgacaagactgaaatgttactgaaaacaatggtgccatatgctccaaagacatttccccaagataactgccaaagagtttttgaggaggacaatgatcatttattatgtaggagccttgatatctctgcaaaatagaattaatacagctcaaatggagtagtaaccaagcttttctgcccaggaagtaacaaacatcactacgaacatgagagtacaagaggaaactttcataatgcattttttcattcatacattcattcaataaacattagccaagctaatgtcccaagccactgtgccaggtattaacaatataacaacaataaaagacacagtccttcctctcaaggtgttcagtctagtagggaagatgattattcattaaaatttttggtgcatcagaatcatgaggagcttgtcaaaaatgtaaattcctgcctatgttctcagatattctggttaggtcaggagtgggaacccaaaatcaattcttttaacaaacactaaaggtgattctaacacaggcggtgtgaggacc

SEQ ID NO: 15 - DNA, 280 bp, Homo sapiens
cgaggtgggccacccgtgtctggtctgagatttttaaatgaggattacattatcctatttataatattcctattctaatctattgtattcttacaattaaatgtatcaaataattcttaaaaacattattagaaacaaactgcctaataccttataagactaaaaaaatcaccaagatgaaactgtattatgactctcaatatttaaacatttaaaaaaatgttagtgtttgttaagcaccaatcttaactatttcacctgcccgggcggccgctcgagg

SEQ ID NO: 16 - DNA, 2041 bp, Homo sapiens
ccccccgcagaactcccccctggaataggatttttaaaacccttgacaattagaaatcctatagaggttagcattttttaggtaaaaatatggttgcccctacagggatcatgcaacttccttaaaaccaattcagcacatatgtataaagaaccctttttaaaaacatttgtacttgaaatacagacacagtgatgctgaagacactaaacaaaaactgaaaagtactataccttgataaattttgttattgccttctttagagactttataatctctagttgattttcaaggacttgaatttaataatggggtaattacacaagacgtaaaggattttttaaaaacaagtatttttttttacctctagcatcaattcttttataaagaatgctaaataaattacattttttgttcagtaaaactgaagatagaccatttaaatgcttctaccaaatttaacgcagcttaattagggaccaggtacatattttcttctgaacatttttggtcaagcatgtctaaccataaaagcaaatggaattttaagaggtagattttttttccatgatgcattttgttaataaatgtgtcaagaaaataaaaacaagcactgagtgtgttctcttgaagtataagggtctaatgaaaaataaaagatagatatttgttatagtctgacattttaacagtcatagtattagacgtttcgtgaccagtgcattttggactctctcaggatcaaaatacgagtctgccaactgtattaaatcctcctccaccccctccaccagttggtccacagcttcctggtgggtcgttgtcatcaaatccattgggccgaaatgaacatgaagcagatgcagcttggagggcccgggctcgagcattcaactcttgttcctgtaaatatagtttattgtcttttgttatagcatccataagttctttctgtagaggtgggtctccatttatccagagtccactggttgggttattaccacttaaaccattagtactatgctgttttttatacaaaagcacataagctgtgtcctttggaaacctgctcgtaattttctggactgactgaaatgaagtaaatgtcactctactgtcattaaataaaaacccattcttttgacatttccttattttccaaatcctgttcaaaaactgcactgggactatctctccctagtaaatgactctgggaggatgctaatgccagagcctcagactggtggtacatctgatatgaagagtctgtacttgtgatatttctggcataagaatagtaatgcccactttcagaggatataccagagtgaaccacaacggaacttaatagatagggcaccaattttgtgcaggaagcttcatcagtccctgaaggctttaattttttagcaaggttctcactaagatcagtgaagtcaacatctacagaccaactttctgacaatgaagagaaagaagtaattcttctaactggcaactccaaaaccagtggccagtgatacattgtctaaaattttccttctcacatgatacttctgatcatatgaaaatctcaggagagtaagaataaggtattcaggttcctccgtgatttgcatagttttctcagcattttgcagagaggcacagttttcacaataatattggttatcaccagtaagaatctctggagcccaaaaaataatttagtaagtcagttactgaaggtgtggtttcacctcccggtttctgaggtacatctttattaacaagaatcttgttagattcgttagggacagaagtgttttcagaacagtaaaactcattaggaggactgcctatggttttttcattcacaagtgagtcacagatgaaggcagctgttgttggattataaactactggctcttctgaaggaccgggtacagacgcttgcattagaccaccatcttgtatactgggtgatgatgctggatcttggacagacatgttttccaaagaagaggaagcacaaaacgcaagcgaaagatctgtaaaggct

SEQ ID NO: 17 - DNA, 235 bp, Homo sapiens
cgccccgggcaggtgtcaggggttccaaaccagcctggggaaacacagcgtagacccctcacctctacaaataaaaaattaaaaaattagccaggtgtggcagcgaacaactgtagtctcagatactcaggagactgagctggaaaggatcacttgagcccaagaagttcaaggttacagtgggccacgatcatgtcattacactccagcttgggtgacaaaatgagactgtcta

SEQ ID NO: 18 - DNA, 2732 bp, Homo sapiens
gtgtggagtttcagctgctattgactataagagctatggaacagaaaaagcttgctggcttcatgttgataactactttatatggagcttcattggacctgttaccttcattattctgctaaatattatcttcttggtgatcacattgtgcaaaatggtgaagcattcaaacactttgaaaccagattctagcaggttggaaaacattaagtcttgggtgcttggcgctttcgctcttctgtgtcttcttggcctcacctggtcctttgggttgctttttattaatgaggagactattgtgatggcatatctcttcactatatttaatgctttccagggagtgttcattttcatctttcactgtgctctccaaaagaaagtacgaaaagaatatggcaagtgcttcagacactcatactgctgtggaggcctcccaactgagagtccccacagttcagtgaaggcatcaaccaccagaaccagtgctcgctattcctctggcacacagagtcgtataagaagaatgtggaatgatactgtgagaaaacaatcagaatcttcttttatctcaggtgacatcaatagcacttcaacacttaatcaaggtggcataaatcttaatatattattacaggactgacatcacatggtctgagagcccatcttcaagatttatatcatttagaggacattcactgaacaatgccagggatacaagtgccatggatactctaccgctaaatggtaattttaacaacagctactcgctgcacaagggtgactataatgacagcgtgcaagttgtggactgtggactaagtctgaatgatactgcttttgagaaaatgatcatttcagaattagtgcacaacaacttacggggcagcagcaagactcacaacctcgagctcacgctaccagtcaaacctgtgattggaggtagcagcagtgaagatgatgctattgtggcagatgcttcatctttaatgcacagcgacaacccagggctggagctccatcacaaagaactcgaggcaccacttattcctcagcggactcactcccttctgtaccaaccccagaagaaagtgaagtccgagggaactgacagctatgtctcccaactgacagcagaggctgaagatcacctacagtcccccaacagagactctctttatacaagcatgcccaatcttagagactctccctatccggagagcagccctgacatggaagaagacctctctccctccaggaggagtgagaatgaggacatttactataaaagcatgccaaatcttggagctggccatcagcttcagatgtgctaccagatcagcaggggcaatagtgatggttatataatccccattaacaaagaagggtgtattccagaaggagatgttagagaaggacaaatgcagctggttacaagtctttaatcatacagctaaggaattccaagggccacatgcgagtattaataaataaagacaccattggcctgacgcagctccctcaaactctgcttgaagagatgactcttgacctgtggttctctggtgtaaaaaagatgactgaaccttgcagttctgtgaatttttataaaacatacaaaaactttgtatatacacagagtatactaaagtgaattatttgttacaaagaaaagagatgccagccaggtattttaagattctgctgctgtttagagaaattgtgaaacaagcaaaacaaaactttccagccattttactgcagcagtctgtgaactaaatttgtaaatatggctgcaccatttttgtaggcctgcattgtattatatacaagacgtaggctttaaaatcctgtgggacaaatttactgtaccttactattcctgacaagacttggaaaagcaggagagatattctgcatcagtttgcagttcactgcaaatcttttacattaaggcaaagattgaaaacatgcttaaccactagcaatcaagccacaggccttatttcatatgtttcctcaactgtacaatgaactattctcatgaaaaatggctaaagaaattatattttgttctattgctagggtaaaataaatacatttgtgtccaactgaaatataattgtcattaaaataattttaaagagtgaagaaaatattgtgaaaagctcttggttgcacatgttatgaaatgttttttcttacactttgtcatggtaagttctactcattttcacttcttttccactgtatacagtgttctgctttgacaaagttagtctttattacttacatttaaatttcttattgccaaaagaacgtgttttatggggagaaacaaactctttgaagccagttatgtcatgccttgcacaaaagtgatgaaatctagaaaagattgtgtgtcacccctgtttattcttgaacagagggcaaagagggcactgggcacttctcacaaactttctagtgaacaaaaggtgcctattcttttttaaaaaaataaaataaaacataaatattactcttccatattccttctgcctatatttagtaattaatttattttatgataaagttctaatgaaatgtaaattgtttcagcaaaattctgcttttttttcatccctttgtgtaaacctgttaataatgagcccatcactaatatccagtgtaaagtttaacacggtttgacagtaaataaatgtgaattttttcaagttaaaaaaaaaaaa

SEQ ID NO: 19 - DNA, 276 bp, Homo sapiens
ctccctaaatgattttaaaataaattggataaacatatgatataaagtgggtactttagaaaccgcctttgcatattttttatgtacaaatctttgtatacaattccgatgttccttatatattccctatatagcaaaccaaaaccaggacctcccaactgcatgcctcaagtccctgtggagcactctggcaactggatggccctacttgctttctgacaaaatagctggaaaggaggagggaccaattaaatacctcggccgcgaccacgctgg

SEQ ID NO: 20 - DNA, 2361 bp, Homo sapiens
attgtaccagccttgatgaacgtgggccctgcttcgcttttgagggccataagctcattgcccactggtttagaggctaccttatcattgtctcccgtgaccggaaggtttctcccaagtcagagtttaccagcagggattcacagagctccgacaagcagattctaaacatctatgacctgtgcaacaagttcatagcctatagcaccgtctttgaggatgtagtggatgtgcttgctgagtggggctccctgtacgtgctgacgcgggatgggcgggtccacgcactgcaggagaaggacacacagaccaaactggagatgctgtttaagaagaacctatttgagatggcgattaaccttgccaagagccagcatctggacagtgatgggctggcccagattttcatgcagtatggagaccatctctacagcaagggcaaccacgatggggctgtccagcaatatatccgaaccattggaaagttggagccatcctacgtgatccgcaagtttctggatgcccagcgcattcacaacctgactgcctacctgcagaccctgcaccgacaatccctggccaatgccgaccataccaccctgctcctcaactgctataccaagctcaaggacagctcgaagctggaggagttcatcaagaaaaagagtgagagtgaagtccactttgatgtggagacagccatcaaggtcctccggcaggctggctactactcccatgccctgtatctggcggagaaccatgcacatcatgagtggtacctgaagatccagctagaagacattaagaattatcaggaagcccttcgatacatcggcaagctgccttttgagcaggcagagagcaacatgaagcgctacggcaagatcctcatgcaccacataccagagcagacaactcagttgctgaagggactttgtactgattatcggcccagcctcgaaggccgcagcgatagggaggccccaggctgcagggccaactctgaggagttcatccccatctttgccaataacccgcgagagctgaaagccttcctagagcacatgagtgaagtgcagccagactcaccccaggggatctacgacacactccttgagctgcgactgcagaactgggcccacgagaaggatccacaggtcaaagagaagcttcacgcagaggccatttccctgctgaagagtggtcgcttctgcgacgtctttgacaaggccctggtcctgtgccagatgcacgacttccaggatggtgtcctttacctttatgagcaggggaagctgttccagcagatcatgcactaccacatgcagcacgagcagtaccggcaggtcatcagcgtgtgtgagcgccatggggagcaggacccctccttgtgggagcaggccctcagctacttcgctcgcaaggaggaggactgcaaggagtatgtggcagctgtcctcaagcatatcgagaacaagaacctcatgccacctcttctagtggtgcagaccctggcccacaactccacagccacactctccgtcatcagggactacctggtccaaaaactacagaaacagagccagcagattgcacaggatgagctgcgggtgcggcggtaccgagaggagaccacccgtatccgccaggagatccaagagctcaaggccagtcctaagattttccaaaagaccaagtgcagcatctgtaacagtgccttggagttgccctcagtccacttcctgtgtggccactccttccaccaacactgctttgagagttactcggaaagtgatgctgactgccccacctgcctccctgaaaaccggaaggtcatggatatgatccgggcccaggaacagaaacgagatctccatgatcaattccagcatcagctcaagtgctccaatgacagcttttctgtgattgctgactactttggcagaggtgttttcaacaaattgactctgctgaccgaccctcccacagccagactgacctccagcctggaggctgggctgcaacgcgacctactcatgcactccaggaggggcacttaagcagcctggaggaagatgtgggcaacagtggaggaccaagagaacagacacaatgggacctgggcgggcgttacacagaaggctggctgacatgcccagggctccactctcatctaatgtcacagccctcacaagactaaagcggaactttttcttttccctggccttccttaattttaagtcaagcttggcaatcccttcctctttaactaggcaggtgttagaatcatttccagattaatgggggggaaggggaacctcaggcaaacctcctgaagttttggaaaaaaaagctggtttc

SEQ ID NO: 21 - DNA, 179 bp, Homo sapiens
aggtgttagatgctcttgaaaaagaaactgcatctaagctgtcagaaatggattcttttaacaatcaactaaaggaactgagagaaacctacaacacacagcagttagcccttgaacagctttataagatcaacgtgacaagttgaaggaaattgaaaggaaaaaattagaactaatgc

SEQ ID NO: 22 - DNA, 905 bp, Homo sapiens
ttttttttttttctttaaccgtgtggtctttatttcagtgccagtgttacagatacaacacaaatgttccagttagaaggaattcaaacggaatgccaaggtccaagccaggctcaagaaataaaaagggaggtttggagtaatagataagatgactccaatactcactcttcctaagggcaaaggtacttttgatacagagtctgatctttgaaactggtgaactcctcttccacccattaccatagttcaaacaggcaagttatgggcttaggagcactttaaaatttgtggtgggaatagggtcattaataactatgaatatatcttttagaaggtgaccattttgcactttaaagggaatcaattttgaaaatcatggagactattcatgactacagctaaagaatggcgagaaaggggagctggaagagccttggaagtttctattacaaatagagcaccatatccttcatgccaaatctcaacaaaagctctttttaactccatctgtccagtgtttacaaataaactcgcaaggtctgaccagttcttggtaacaaacatacatgtgtgtgtctgtgtgtatacagcaatgcacagaaaaggctaccaggagcctaatgcctctttcaaacattgggggaaccagtagaaaaaggcagggctccctaatgtccattattacatttccattccgaatgccagatgttaaaagtgcctgaagatggtaacccagctagtgaggaataaataccccaccttgcccagtccacagagaaacaacagtagaaagaaggggcaactctttgctgcagagacaaagtgagtgttttttcgccatggattgcagtcctctcctccagaccagctgcttatttcctcaggggcccagggaatgttga

SEQ ID NO: 23 - DNA, 2134 bp, Homo sapiens
ggtctcttctttcctttttttttttccaaaagtgttcttttatttctagtaacatatattgtataaatactctattttatatgcacttccacaaaagcgatataatttaaaagtttttttcattagaaataaatgtataaaaataaatatgttattataggcatttattactaactatagtccttcttggaaggaacacccaaaccaatacttataaagtacatgtaatttatagtaacatattttactatatacatatggaaaaaatcatattctcacagaagagctgaacagacattcaccaggatacgactgttggaccagctgctggagatggacctgctacccctcagcagcctccccaccacaagacaagtgatctcaatgtccccaaacctgtgggaccctgttctacacacctcatttttgttccggcgtttcatcctccttgtgtgattgtactgattttcatgagacacaagttacttctttacatccatattcccaaagcagggttacatggtaggaaagaaaggaagttggaggtactaagctcattgtgtctcctctagcttttaccagcatctaatgcttcactgctttttttccattgtagactttaatgcacttgaataaatacatggagttgttttttcctcaaaatgaattacacaaataaagactgagatggtccaaaaaaggaaagaggaagccatttgcgttatttcacgttgctgagcctttctctcatgttgaacaatctgaagttttaattctcggtagaaataatgtataaacattctctgaaaccatagcagccataaacagtgctggtcaaagatcctatttgtactcctttctccccccattgttagtgaggtaaagtaaaacaggtcttagtaaaatctcacttttctcctacttttcatttcccaacccccatgatactaagtatttgataagtaccaggaaacaggggttgtaatagttctaactttttttgacaattgctttgttttttctaaacttgtaatagatgtaacaaaagaaataataataataatgcccggggctttattatgctatatcactgctcagaggttaataatcctcactaactatcctatcaaatttgcaactggcagtttactctgatgattcaactccttttctatctacccccataatcccaccttactgatacacctcactggttactggcaagatacgctggatccctccagccttcttgctttccctgcaccagcccttcctcactttgccttgccctcaaagctaacaccacttaaaccacttaactgcattctgccattgtgcaaaagtctatgaaatgtttaggtttctttaaaggatcacagctctcatgagataacacccctccatcatgggacagacacttcaagcttctttttttgtaacccttcccacaggtcttagaacatgatgaccactcccccagctgccactgggggcagggatggtctgcacaaggtctggtgctggctggcttcacttcctttgcacactcggaagcaggctgtccattaatgtctcggcattctaccagtcttctctgccaacccaattcacatgacttagaacattcgccccactcttcaatgacccatgctgaaaaagtggggatagcattgaaagattccttcttcttctttacgaagtaggtgtatttaattttaggtcgaagggcattgcccacagtaagaacctggatggtcaagggctctttgagagggctaaagctgcgaattctttccaatgccgcagaggagccgctgtacctcaagacaacacctttgtacataatgtcttgctctaaggtggacaaagtgtagtcaccattaagaatatatgtgccatcagcagctttgatggcaagaaagctgccattgttcctggatcccctctggttccgctgtttcacttcgatgttggtggctccagttggaattgtgatgatatcatgatatccaggttttgcactagtaactgatcctgatatttttttacaagtagatccatttcccccgcaaacaccacatttatcaaacttctttttggagtctatgatgcgatcacaaccagcttttacaca

SEQ ID NO: 24 - DNA, 1626 bp, Homo sapiens
ggacaatttctagaatctatagtagtatcaggatatattttgctttaaaatatattttggttattttgaatacagacattggctccaaattttcatctttgcacaatagtatgacttttcactagaacttctcaacatttgggaactttgcaaatatgagcatcatatgtgttaaggctgtatcatttaatgctatgagatacattgttttctccctatgccaaacaggtgaacaaacgtagttgttttttactgatactaaatgttggctacctgtgattttatagtatgcacatgtcagaaaaaggcaagacaaatggcctcttgtactgaatacttcggcaaacttattgggtcttcattttctgacagacaggatttgactcaatatttgtagagcttgcgtagaatggattacatggtagtgatgcactggtagaaatggtttttagttattgactcagaattcatctcaggatgaatcttttatgtctttttattgtaagcatatctgaatttactttataaagatggttttagaaagctttgtctaaaaatttggcctaggaatggtaacttcattttcagttgccaaggggtagaaaaataatatgtgtgttgttatgtttatgttaacatattattaggtactatctatgaatgtatttaaatatttttcatattctgtgacaagcatttataatttgcaacaagtggagtccatttagcccagtgggaaagtcttggaactcaggttacccttgaaggatatgctggcagccatctctttgatctgtgcttaaactgtaatttatagaccagctaaatccctaacttggatctggaatgcattagttatgccttgtaccattcccagaatttcaggggcatcgtgggtttggtctagtgattgaaaacacaagaacagagagatccagctgaaaaagagtgatcctcaatatcctaactaactggtcctcaactcaagcagagtttcttcactctggcactgtgatcatgaaacttagtagaggggattgtgtgtattttatacaaatttaatacaatgtcttacattgataaaattcttaaagagcaaaactgcattttatttctgcatccacattccaatcatattagaactaagatatttatctatgaagatataaatggtgcagagagactttcatctgtggattgcgttgtttcttagggttcctagcactgatgcctgcacaagcatgtgatatgtgaaataaaatggattcttctatagctaaatgagttccctctggggagagttctggtactgcaatcacaatgccagatggtgtttatgggctatttgtgtaagtaagtggtaagatgctatgaagtaagtgtgtttgttttcatcttatggaaactcttgatgcatgtgcttttgtatggaataaattttggtgcaatatgatgtcattcaactttgcattgaattgaattttggttgtatttatatgtattatacctgtcacgcttctagttgcttcaaccattttataaccatttttgtacatattttacttgaaaatattttaaatggaaatttaaataaacatttgatagtttacataataaaaaaaaaaa

SEQ ID NO: 25 - DNA, 1420 bp, Homo sapiens
gttcagcattgtttctgcttctgaaatctgtatagtacactggtttgtaatcattatgtcttcattgaaatccttgctacttctcttcctcctcaatgaaagacacgagagacaagagcgacacaagcttaagaaaaacgagcaaggaagagtatcttcattattctcattttctctgagttggaaacaaaaacatgaaggactccaactagaagacagatatttacatttaaatagattagtgggaaaactttaagagtttccacatattagttttcattttttgagtcaagagactgctccttgtactgggagacactagtagtatatgtttgtaatgttactttaaaattatctttttattttataaggcccataaatactggttaaactctgttaaaagtgggccttctatcttggatggtttcactgccatcagccatgctgatatattagaaatggcatccctatctacttactttaatgcttaaaattatacataaaatgctttatttagaaaacctacatgatacagtggtgtcagccttgccatgtatcagtttcacttgaaatttgagaccaattaaatttcaactgtttagggtggagaaagaggtactggaaaacatgcagatgaggatatcttttatgtgcaacagtatcctttgcatgggaggagagttactcttgaaaggcaggcagcttaagtggacaatgttttgtatatagttgagaattttacgacacttttaaaaattgtgtaattgttaaatgtccagttttgctctgttttgcctgaagttttagtatttgttttctaggtggacctctgaaaaccaaaccagtacctggggaggttagatgtgtgtttcaggcttggagtgtatgagtggttttgcttgtattttcctccagagattttgaactttaataattgcgtgtgtgtttttttttttttaagtggctttgtttttttttctcaagtaaaattgtgaacatatttcctttataggggcagggcatgagttagggagactgaagagtattgtagactgtacatgtgccttcttaatgtgtttctcgacacattttttttcagtaacttgaaaattcaaaagggacatttggttaggttactgtacatcaatctatgcataaatggcagcttgttttcttgagccactgtctaaattttgtttttatagaaattttttatactgattggttcatagatggtcagttttgtacacagactgaacaatacagcactttgccaaaaatgagtgtagcattgtttaaacattgtgtgttaacacctgttctttgtaattgggttgtggtgcattttgcactacctggagttacagttttcaatctgtcagtaaataaagtgtcctttaacttcaaaaaaaaaaaaaaa

SEQ ID NO: 26 - DNA, 689 bp, Homo sapiens
aaacaaacaaaaaaaaagttagtactgtatatgtaaatactagcttttcaatgtgctatacaaacaattatagcacatccttccttttactctgtctcacctcctttaggtgagtacttccttaaataagtgctaaacatacatatacggaacttgaaagctttggttagccttgccttaggtaatcagcctagtttacactgtttccagggagtagttgaattactataaaccattagccacttgtctctgcaccatttatcacaccaggacagggtctctcaacctgggcgctactgtcatttggggccaggtgattcttccttgcaagggctgtcctgtacctgcccgggcggccgctcgaagcgtggtcgcggccgaggtactgaaaggaccaaggagctctggctgccctcaggaattccaaatgaccgaaggaacaaagcttcagggctctgggtggtgtctcccactattcaggaggtggtcggaggtaacgcagcttcatttcgtccagtcctttccagtatttaaagttgttgtcaagatgctgcattaaatcaggcaggtctacaaaggcatcccaagcatcaaacatgtctgtgatgaagtaatcaatgaaacaccggaacctccgaccacctcctgaatagtgggagacacacccagagcctgaagtttgtccttcg

SEQ ID NO: 27 - DNA, 471 bp, Homo sapiens
tcccagcggcatgaagtttgagattggccaggccctgtacctgggcttcatctccttcgtccctctcgctcattggtggcaccctgctttgcctgtcctgccaggacgaggcaccctacaagccctaacccaggccccgcccagggccaccacgaccactgcaaacaccgcacctgcctaccagccaccagctgcctacaaagacaatcgggccccctcagtgacctcggccaccacagcgggtacaggctgaacgactacgtgtgagtccccacagcctgcttctcccctgggctgctgtgggctggttcccggcgggactgtcaatggaggcaggggttccagcacaaagtttacttctgggcaatttttgtatccaaggaaataatgtgaatgcgaggaaatgtctttagagcacagggacagagggggaaataagaggaggagaaagctctctataccaaagactga

SEQ ID NO: 28 - DNA, 929 bp, Homo sapiens
ggtgaactcagtgcattgggccaatggttcgacacaggctctgccagccacaaccatcctgctgcttctgacggtttggctgctggtgggctttcccctcactgtcattggaggcatctttgggaagaacaacgccagcccctttgatgcaccctgtcgcaccaagaacatcgcccgggagattccaccccagccctggtacaagtctactgtcatccacatgactgttggaggcttcctgcctttcagtgccatctctgtggagctgtactacatctttgccacagtatggggtcgggagcagtacactttgtacggcatcctcttctttgtcttcgccatcctgctgagtgtgggggcttgcatctccattgcactcacctacttccagttgtctggggaggattaccgctggtggtggcgatctgtgctgagtgttggctccaccggcctcttcatcttcctctactcagttttctattatgcccggcgctccaacatgtctggggcagtacagacagtagagttcttcggctactccttactcactggttatgtcttcttcctcatgctgggcaccatctcctttttttcttccctaaagttcatccggtatatctatgttaacctcaagatggactgagttctgtatggcagaactattgctgttctctccctttcttcatgccctgttgaactctcctaccagcttctcttctgattgactgaattgtgtgatggcattgttgccttccctttttccctttgggcattccttccccagagagggcctggaaattataaatctctatcacataaggattatatatttgaactttttaagttgcctttagttttggtcctgatttttctttttacaattaccaaaataaaatttattaagaaaaagaaaaaaaaaaaaaaaaaaa

SEQ ID NO: 29 - DNA, 1775 bp, Homo sapiens
gaacgtgatgggaactttgggaggatgtctgagaaaatgtccgaagggattttggccaacaccagaaaacgccaatgtcctaggaattccctcccaaaatgcttcccaaaaaattactcattgacaattcaaattgcacttggctggcggcagcccgggcggccttcagtccgtgtggggcgcccgcgtggccttctcctcgtaggactccccaaactcgttcactctgcgtttatccacaggataaagccaccgctggtacaggtagaccagaaacaccacgtcgtcccggaagcaggccagccggtgagacgtgggcatggtgatgatgaaggcaaagacgtcatcaatgaaggtgttgaaagccttgtaggtgaaggccttccagggcagatgtgccactgacttcaacttgtagttcacaaagagctggggcagcatgaagaggaaaccaaaggcatagaccccgttgacgaagctgttgattaaccaggagtaccagctcttatatttgatattcaggagtgaatagacagcacccccgacacagagagggtacagcaggtatgacaagtacttcatggcctgagtatcgtactcctcggttttcctctcagattcgctgtaagtgccaaactgaaattcgggcatcaggcctctccaaaaaatagtcatcttcaatgccttcttcactttccacagctcaatggcggctccaacacccgccgggaccagcaccagcaggctcgtctgctcgtccagcaggaacagaaagatgaccacggtgctgaagcagcgccagagcactgccttggtggacatgccgatcatgctcttcttcttcttccagaaactgatgtcatttttaaaggccaggaaatcaaagagaagatggaacgctgcgacaaagaaggtcagcgccaggaagtataagttggtatctacaaaaattcctttcacctcatcagcatctttctctgaaaacccgaactgctgcagggagtacacggcgtcctgcatgtggatccagaagcgcagccgccccagtgagaccttgtcgtaggacacggtgaggggcagctcggtggtggagcggtttatgaccatcaggtccttcacgcggttgctgagctggtcgatgaacaggatgggcaggtaatgcacggttttccccagctggatcatcttcatgtaccgatgcacatcggcaggcagggaggacccgtcaaagacaaagttgtccgccatcacgttcagcgccagccgcggtcgccagtgggacactggctcatccagggcactcgtcggcttcttctccgcctcgatctgctgtgtatcagactccccggtgagcaggttgatttcttctggcttggggaccatgtaggtggtcagaggactgaccaggtgcacctgcttcccgtcgtgccacggcaggaccccagcgtgatggaggaagatgtaggcatacagcgtcccattgtttctcgttttctttggtacagaaacattaactgtcctttcaaatttggactccacatcaaagtcttccacattcaagaccaggtcgatgttgttctcagcacccaggtgggacctcgtcgtggtgtacacgctcagctgcagcttgggccgccgcgccaggtagggctggatgcagttggcgtcgccggagcacgggcgggtgtagacgatgccgtacatgacccagcaggtgtgcaccacgtagaccacgaacacgcccaccaccaagctggtgaaggagctgcggcccc

SEQ ID NO: 30 - DNA, 1546 bp, Homo sapiens
aaaataagtaggaatgggcagtgggtattcacattcactacaccttttccatttgctaataaggccctgccaggctgggagggaattgtccctgcctgcttctggagaaagaagatattgacaccatctacgggcaccatggaactgcttcaagtgaccattctttttcttctgcccagtatttgcagcagtaacagcacaggtgttttagaggcagctaataattcacttgttgttactacaacaaaaccatctataacaacaccaaacacagaatcattacagaaaaatgttgtcacaccaacaactggaacaactcctaaaggaacaatcaccaatgaattacttaaaatgtctctgatgtcaacagctacttttttaacaagtaaagatgaaggattgaaagccacaaccactgatgtcaggaagaatgactccatcatttcaaacgtaacagtaacaagtgttacacttccaaatgctgtttcaacattacaaagttccaaacccaagactgaaactcagagttcaattaaaacaacagaaataccaggtagtgttctacaaccagatgcatcaccttctaaaactggtacattaacctcaataccagttacaattccagaaaacacctcacagtctcaagtaataggcactgagggtggaaaaaatgcaagcacttcagcaaccagccggtcttattccagtattattttgccggtggttattgctttgattgtaataacactttcagtatttgttctggtgggtttgtaccgaatgtgctggaaggcagatccgggcacaccagaaaatggaaatgatcaacctcagtctgataaagagagcgtgaagcttcttaccgttaagacaatttctcatgagtctggtgagcactctgcacaaggaaaaaccaagaactgacagcttgaggaattctctccacacctaggcaataattacgcttaatcttcagcttctatgcaccaagcgtggaaaaggagaaagtcctgcagaatcaatcccgacttccatacctgctgctggactgtaccagacgtctgtcccagtaaagtgatgtccagctgacatgcaataatttgatggaatcaaaaagaaccccggggctctcctgttctctcacatttaaaaattccattactccatttacaggagcgttcctaggaaaaggaattttaggaggagaatttgtgagcagtgaatctgacagcccaggaggtgggctcgctgataggcatgactttccttaatgtttaaagttttccgggccaagaatttttatccatgaagactttcctacttttctcggtgttcttatattacctactgttagtatttattgtttaccactatgttaatgcagggaaaagttgcacgtgtattattaaatattaggtagaaatcataccatgctactttgtacatataagtattttattcctgctttcgtgttacttttaataaataactactgtactcaatactctaaaaatactataacatgactgtgaaaatggcaaaaaaa

SEQ ID NO: 31 - DNA, 750 bp, Homo sapiens
cacttgggcacccccattttctaaaaaaatggaaatctggagggcaaaaaaggtgtgctgaagggaagtgcctctgatggcccaaaaaccttcttccaaactagtgtaggaatggaatggatagcaaatggatcctttttggcctcctttggagcatgccttccctatcttatccttggccccactaaagcagaacgttacggatatttctgtttttgccattggatgcctatctggccaaacagcctttccctaattggaaaatgcagtcctgtttaaaacctttgatttacgactacttgtacatgcttgctcattacaattttgacattttttacatagtgaagaccccaaacatatcagtgaaacatgacaagatcataaagaacagtatcatattattatttagtcgcttttacagtggcaagccaattttgaaatatctcatttaaaactcagacccaattcactgagttatacttttaatagcttcctcagcacactatttcccatgcattaaatatgataaaataatctatcactgcccatcggtcttgtaaaaaggaagtctgaatacagagcccacaacactaaaattgtttttctagctacaaagtatagcatcatcaacacagacacgatttggactccctgacaggtggattggaaaacggtgtttaaagagaagagaacattttaacataaatgtcattaagaatcccaaaggccttatttgtcaccaccgtcccg

SEQ ID NO: 32 - DNA, 1620 bp, Homo sapiens
gcaattcccccctcccactaaacgactcccagtaattatgtttacaacccattggatgcagtgcagccattcataagaaccttggtgccccagaaaaatctgtcctttttggtaccaaacctgaggtcttttggaagataatgtagaaaaccactacctattgaaggcctgttttggctaatctgtgcaaactctgatgatacctgccttatgtggattcttttccacactgctttcatttttaagtataaagacttagaaaactagaataatgcttttacaaataattaaaagtatgtgatgttctgggttttttccttctttttagaaccccgcctccatttaaaaaattaaaaaaaaaaaaaaaacttttaacatttaaaaaataaaaattaacaaaatttcacttattccaggacacgctggcatttggactcaatgaaaagggcacctaaagaaaataaggctgactgaatgttttccataattttcacacaataacagtccctttctatccagcttgccttccatttatctctagggttagcttttcaggcaacatccttggtcattgcccagaaagtacctgagctatcagtgattggaatggcacaggaaaccgaatcacatgggtgccctccccttggttttcaagtatcttggagttgtgcacaaaaattaggtcatgccttcagtgtcttgttctttaaacctaccctttgacaatcaggtgctaatgattgtatactattaaaaccagcacataagtattgtaaatgtgtgttcctcctaggttggaagaaatgtctttccttctatctgggtcctgttaaagcgggtgtcagttgtgtcttttcacctcgatttgtgaattaatagaattggggggagaggaaatgatgatgtcaattaagtttcaggtttggcatgatcatcattctcgatgatattctcactttgtcgcaaatctgcccttatcgtaagaacaagtttcagaattttccctccactatacgactccagtattatgtttacaatccattggatgagtgcagcattataagaccttggtgcccagaaaaatctgtcctttttggtaccaaacctgaggtcttttggaagataatgtagaaaaccactacctattgaaggcctgttttggctaatctgtgcaaactctgatgatacctgcttatgtggattcttttccacactgctttcatttttaagtataaagacttagaaaactagaataatgcttttacaaataattaaaagtatgtgatgttctgggttttttccttctttttagaaccctgtatttaaacaagccttctttttaagtcttgtttgaaatttaagtctcagatcttctggataccaaatcaaaaacccaacgcgtaaaacagggcagtatttgtgttcctaattttaaaaagctttatgtatactctataaatatagatgcataaacaacacttccccttgagtagcacatcaacatacagcattgtacattacaatgaaaatgtgtaacttaagggtattatatatataaatacatatatacctttgtaacctttatactgtaaataaaaaagttgctttagtcaaaaaaaa

SEQ ID NO: 33 - DNA, 2968 bp, Homo sapiens
gaaaaagtagaaggaaacacagttcatatagaagtaaaagaaaaccctgaagaggaggaggaggaggaagaagaggaagaagaagatgaagaaagtgaagaggaggaggaagaggagggagaaagtgaaggcagtgaaggtgatgaggaagatgaaaaggtgtcagatgagaaggattcagggaagacattagataaaaagccaagtaaagaaatgagctcagattctgaatatgactctgatgatgatcggactaaagaagaaagggcttatgacaaagcaaaacggaggattgagaaacggcgacttgaacatagtaaaaatgtaaacaccgaaaagctaagagcccctattatctgcgtacttgggcatgtggacacagggaagacaaaaattctagataagctccgtcacacacatgtacaagatggtgaagcaggtggtatcacacaacaaattggggccaccaatgttcctcttgaagctattaatgaacagactaagatgattaaaaattttgatagagagaatgtacggattccaggaatgctaattattgatactcctgggcatgaatctttcagtaatctgagaaatagaggaagctctctttgtgacattgccattttagttgttgatattatgcatggtttggagccccagacaattgagtctatcaaccttctcaaatctaaaaaatgtcccttcattgttgcactcaataagattgataggttatatgattggaaaaagagtcctgactctgatgtggctgctactttaaagaagcagaaaaagaatacaaaagatgaatttgaggagcgagcaaaggctattattgtagaatttgcacagcagggtttgaatgctgctttgttttatgagaataaagatccccgcacttttgtgtctttggtacctacctctgcacatactggtgatggcatgggaagtctgatctaccttcttgtagagttaactcagaccatgttgagcaagagacttgcacactgtgaagagctgagagcacaggtgatggaggttaaagctctcccggggatgggcaccactatagatgtcatcttgatcaatgggcgtttgaaggaaggagatacaatcattgttcctggagtagaagggcccattgtaactcagattcgaggcctcctgttacctcctcctatgaaggaattacgagtgaagaaccagtatgaaaagcataaagaagtagaagcagctcagggggtaaagattcttggaaaagacctggagaaaacattggctggtttacccctccttgtggcttataaagaagatgaaatccctgttcttaaagatgaattgatccatgagttaaagcagacactaaatgctatcaaattagaagaaaaaggagtctatgtccaggcatctacactgggttctttggaagctctactggaatttctgaaaacatcagaagtgccctatgcaggaattaacattggcccagtgcataaaaaagatgttatgaaggcttcagtgatgttggaacatgaccctcagtatgcagtaattttggccttcgatgtgagaattgaacgagatgcacaagaaatggctgatagtttaggagttagaatttttagtgcagaaattatttatcatttatttgatgcctttacaaaatatagacaagactacaagaaacagaaacaagaagaatttaagcacatagcagtatttccctgcaagataaaaatcctccctcagtacatttttaattctcgagatccgatagtgatgggggtgacggtggaagcaggtcaggtgaaacaggggacacccatgtgtgtcccaagcaaaaattttgttgacatcggaatagtaacaagtattgaaataaaccataaacaagtggatgttgcaaaaaaaggacaagaagtttgtgtaaaaatagaacctatccctggtgagtcacccaaaatgtttggaagacattttgaagctacagatattcttgttagtaagatcagccggcagtccattgatgcactcaaagactggttcagagatgaaatgcagaagagtgactggcagcttattgtggagctgaagaaagtatttgaaatcatctaattttttcacatggagcaggaactggagtaaatgcaatactgtgttgtaatatcccaacaaaaatcagacaaaaaatggaacagacgtatttggacactgatggacttaagtatggaaggaagaaaaataggtgtataaaatgttttccatgagaaaccaagaaacttacactggtttgacagtggtcagttacatgtccccacagttccaatgtgcctgttcactcacctctcccttccccaacccttctctacttggctgctgttttaaagtttgcccttccccaaatttggatttttattacagatctaaagctctttcgattttatactgattaaatcagtactgcagtatttgattaaaaaaaaaaaagcagattttgtgattcttgggacttttttgacgtaagaaatacttctttatttatgcatattcttcccacagtgatttttccagcattcttctgccatatgcctttagggcttttataaaatagaaaattaggcattctgatatttctttagctgctttgtgtgaaaccatggtgtaaaagcacagctggctgctttttactgcttgtgtagtcacgagtccattgtaatcatcacaattctaaaccaaactaccaataaagaaaacagacatccaccagtaagcaagctctgttaggcttccatggttagtggtagcttctctcccacaagttgtcctcctaggacaaggaattatcttaacaaactaaactatccatcacactaccttggtatgccagcacctgggtaacagtaggagattttatacattaatctgatctgtttaatctgatcggtttagtagagattttatacat

SEQ ID NO: 34 - DNA, 6011 bp, Homo sapiens
acggggcgccggacgacccgcacatcttatcctccacgccccactcgcactcggagcgggaccgccccggactccccctcgggccggccactcgaggagtgaggagagaggccgccggcccggcttgagccgagcgcagcaccccccgcgccccgcgccagaagtttggttgaaccgggctgccgggagaaacttttttcttttttccccctctcccgggagagtctctggaggaggaggggaactcccccggcccaaggctcgtgggctcggggtcgcgcggccgcagaaggggcggggtccgcccgcgaggggaggcgcccccggggacccgagaggggggtgaggaccgcgggctgctggtgcggcggcggcagcgtgtgccccgcgcaggggaggcgccgccccgctcccggcccggctgcgaggaggaggcggcggcggcgcaggaggatgtacttggtggcgggggacagggggttggccggctgcgggcacctcctggtctcgctgctggggctgctgctgctgccggcgcgctccggcacccgggcgctggtctgcctgccctgtgacgagtccaagtgcgaggagcccaggaaccgcccggggagcatcgtgcagggcgtctgcggctgctgctacacgtgcgccagccaggggaacgagagctgcggcggcaccttcgggatttacggaacctgcgaccgggggctgcgttgtgtcatccgccccccgctcaatggcgactccctcaccgagtacgaagcgggcgtttgcgaagatgagaactggactgatgaccaactgcttggttttaaaccatgcaatgaaaaccttattgctggctgcaatataatcaatgggaaatgtgaatgtaacaccattcgaacctgcagcaatccctttgagtttccaagtcaggatatgtgcctttcagctttaaagagaattgaagaagagaagccagattgctccaaggcccgctgtgaagtccagttctctccacgttgtcctgaagattctgttctgatcgagggttatgctcctcctggggagtgctgtcccttacccagccgctgcgtgtgcaaccccgcaggctgtctgcgcaaagtctgccagccgggaaacctgaacatactagtgtcaaaagcctcagggaagccgggagagtgctgtgacctctatgagtgcaaaccagttttcggcgtggactgcaggactgtggaatgccctactgttcagcagaccgcgtgtcccccggacagctatgaaactcaagtcagactaactgcagatggttgctgtactttgccaacaagatgcgagtgtctctctggcttatgtggtttccccgtgtgtgaggtgggatccactccccgcatagtctctcgtggcgatgggacacctggaaagtgctgtgatgtctttgaatgtgttaatgatacaaagccagcctgcgtatttaacaatgtggaatattatgatggagacatgtttcgaatggacaactgtcggttctgtcgatgccaagggggcgttgccatctgcttcaccgcccagtgtggtgagataaactgcgagaggtactacgtgcccgaaggagagtgctgcccagtgtgtgaagatccagtgtatccttttaataatcccgctggctgctatgccaatggcctgatccttgcccacggagaccggtggcgggaagacgactgcacattctgccagtgcgtcaacggtgaacgccactgcgttgcgaccgtctgcggacagacctgcacaaaccctgtgaaagtgcctggggagtgttgccctgtgtgcgaagaaccaaccatcatcacagttgatccacctgcatgtggggagttatcaaactgcactctgacacggaaggactgcattaatggtttcaaacgcgatcacaatggttgtcggacctgtcagtgcataaacacccaggaactatgttcagaacgtaaacaaggctgcaccttgaactgtcccttcggtttccttactgatgcccaaaactgtgagatctgtgagtgccgcccaaggcccaagaagtgcagacccataatctgtgacaagtattgtccacttggattgctgaagaataagcacggctgtgacatctgtcgctgtaagaaatgtccagagctctcatgcagtaagatctgccccttgggtttccagcaggacagtcacggctgtcttatctgcaagtgcagagaggcctctgcttcagctgggccacccatcctgtcgggcacttgtctcaccgtggatggtcatcatcataaaaatgaggagagctggcacgatgggtgccgggaatgctactgtctcaatggacgggaaatgtgtgccctgatcacctgcccggtgcctgcctgtggcaaccccaccattcaccctggacagtgctgcccatcatgtgcagatgactttgtggtgcagaagccagagctcagtactccctccatttgccacgcccctggaggagaatactttgtggaaggagaaacgtggaacattgactcctgtactcagtgcacctgccacagcggacgggtgctgtgtgagacagaggtgtgcccaccgctgctctgccagaacccctcacgcacccaggattcctgctgcccacagtgtacagatcaaccttttcggccttccttgtcccgcaataacagcgtacctaattactgcaaaaatgatgaaggggatatattcctggcagctgagtcctggaagcctgacgtttgtaccagctgcatctgcattgatagcgtaattagctgtttctctgagtcctgcccttctgtatcctgtgaaagacctgtcttgagaaaaggccagtgttgtccctactgcataaaagacacaattccaaagaaggtggtgtgccacttcagtgggaaggcctatgccgacgaggagcggtgggaccttgacagctgcacccactgctactgcctgcagggccagaccctctgctcgaccgtcagctgcccccctctgccctgtgttgagcccatcaacgtggaaggaagttgctgcccaatgtgtccagaaatgtatgtcccagaaccaaccaatatacccattgagaagacaaaccatcgaggagaggttgacctggaggttcccctgtggcccacgcctagtgaaaatgatatcgtccatctccctagagatatgggtcacctccaggtagattacagagataacaggctgcacccaagtgaagattcttcactggactccattgcctcagttgtggttcccataattatatgcctctctattataatagcattcctattcatcaatcagaagaaacagtggataccactgctttgctggtatcgaacaccaactaagccttcttccttaaataatcagctagtatctgtggactgcaagaaaggaaccagagtccaggtggacagttcccagagaatgctaagaattgcagaaccagatgcaagattcagtggcttctacagcatgcaaaaacagaaccatctacaggcagacaatttctaccaaacagtgtgaagaaaggcaactaggatgaggtttcaaaagacggaagacgactaaatctgctctaaaaagtaaactagaatttgtgcacttgcttagtggattgtattggattgtgacttgatgtacagcgctaagaccttactgggatgggctctgtctacagcaatgtgcagaacaagcattcccacttttcctcaagataactgaccaagtgttttcttagaaccaaagtttttaaagttgctaagatatatttgcctgtaagatagctgtagagatatttggggtggggacagtgagtttggatggggaaaggggtgggagggtggtgttgggaagaaaaattggtcagcttggctcggggagaaacctggtaacataaaagcagttcagtggcccagaggttatttttttcctattgctctgaagactgcactggttgctgcaaagctcaggcctgaatgagcaggaaacaaaaaaggccttgcgacccagctgccataaccaccttagaactaccagacgagcacatcagaaccctttgacagccatcccaggtctaaagccacaagtttcttttctatacagtcacaactgcagtaggcagtgaggaagccagagaaatgcgatagcggcatttctctaaagcgggttattaaggatatatacagttacactttttgctgcttttattttcttccaagccaatcaatcagccagttcctagcagagtcagcacatgaacaagatctaagtcatttcttgatgtgagcactggagctttttttttttacaacgtgacaggaagaggagggagagggtgacgaacaccaggcatttccaggggctatatttcactgtttgttgttgctttgttctgttatattgttggttgttcatagtttttgttgaagctctagcttaagaagaaactttttttaaaaagactgtttggggattctttttccttattatatactgattctacaaaatagaaactacttcattttaattgtatattattcaagcacctttgttgaagctcaaaaaaaatgatgcctctttaaactttagcaattataggagtatttatgtaactatcttatgcttcaaaaaacaaaagtatttgtgtgcatgtgtatataatatatatatatacatatatatttatacacatacaatttatgttttcctgttgaatgtatttttatgagattttaaccagaacaaaggcagataaacaggcattccatagcagtgcttttgatcacttacaaattttttgaataacacaaaatctcattctacctgcagtttaattggaaagatgtgtgtgtgagagtatgtatgtgtgtgtgtgtgtgtgtgtgtgcgcgcgcacgcacgccttgagcagtcagcattgcacctgctatggagaagggtattcctttattaaaatcttcctcatttggatttgctttcagttggttttcaatttgctcactggccagagacattgatggcagttcttatctgcatcactaatcagctcctggatttttttttttttttttcaaacaatggtttgaaacaactactggaatattgtccacaataagctggaagtttgttgtagtatgcctcaaatataactgactgtatactatagtggtaacttttcaaacagcccttagcacttttatactaattaacccatttgtgcattgagttttcttttaaaaatgcttgttgtgaaagacacagatacccagtatgcttaacgtgaaaagaaaatgtgttctgttttgtaaaggaactttcaagtattgttgtaaatacttggacagaggttgctgaactttaaaaaaaattaatttattattataatgacctaatttattaatctgaagattaaccatttttttgtcttagaatatcaaaaagaaaaagaaaaaggtgttctagctgtttgcatcaaaggaaaaaaagatttattatcaaggggcaatatttttatcttttccaaaataaatttgttaatgatacattacaaaaatagattgacatcagcctgattagtataaattttgttggtaattaatccattcctggcataaaaagtctttatcaaaaaaaattgtagatgcttgctttttgttttttcaatcatggccatattatgaaaatactaacaggatataggacaaggtgtaaatttttttattattattttaaagatatgatttatcctgagtgctgtatctattactcttttactttggttcctgttgtgctcttgtaaaagaaaaatataatttcctgaagaataaaatagatatatggcacttggagtgcatcatagttctacagtttgtttttgttttcttcaaaaaagctgtaagagaattatctgcaacttgattcttggcaggaaataaacattttgagttgaaatcaaaaaaaaaaaaaaaaaa

SEQ ID NO: 35 - DNA, 716 bp, Homo sapiens
gcagtacctggagtgtcctgcagggggaaagcgaaccgggccctgaagtccggggcagtcacccggggctcctgggccgctctgccgggctggggctgagcagcgatcctgctttgtcccagaagtccagagggatcagccccagaacacaccctcctccccgggacgccgcagctttctggaggctgaggaaggcatgaagagtgggctccacctgctggccgactgagaaaagaatttccagaactcggtcctattttacagattgagaaactatggttcaagaagagaggacggggcttgagggaatctcctgattctccttatatgacctcaaactgaccatactaaacagtgtagaaggtctttttaaggctctaaatgtcagggtctcccatcccctgatgcctgacttgtacagtcagtgtggagtagacggtttcctccacccagggttgactcagggggatgatctgggtcccattctggtcttaagaccccaaacaagggttttttcagctccaggatctggagcctctatctggttagtgtcgtaacctctgtgtgcctcccgttaccccatctgtccagtgagctcagcccccatccacctaacagggtggccacagggattactgagggttaagaccttagaactgggtctagcacccgataagagctcaataaatgttgttcctttccacatcaaaaaaaaa

SEQ ID NO: 36 - DNA, 395 bp, Homo sapiens
ccaatacttcattcttcattggtggagaagattgtagacttctaagcattttccaaataaaaaagctatgatttgatttccaacttttaaacattgcatgtcctttgccatttactacattctccaaaaaaaccttgaaatgaagaaggccacccttaaaatacttcagaggctgaaaatatgattattacattggaatcctttagcctatgtgatatttctttaactttgcactttcacgcccagtaaaaccaaagtcagggtaaccaatgtcattttacaaaatgttaaaaccctaattgcagttccttttttaaattattttaaagattacttaacaacattagacagtgcaaaaaaagaagcaaggaaagcattcttaattctaccatcct

SEQ ID NO: 37 - DNA, 134 bp, Homo sapiens
ccctcgagcggccgcccgggcaggtacttttaccaccgaattgttcacttgactttaagaaacccataaagctgcctggctttcagcaacaggcctatcaacaccatggtgagtctccataagggacaccgtgt

SEQ ID NO: 38 - DNA, 644 bp, Homo sapiens
aagcctgttgtcatgggggaggtggtggcgcttggtggccactggcggccgaggtagaggcagtggcgcttgagttggtcgggggcagcggcagatttgaggcttaagcaacttcttccggggaagagtgccagtgcagccactgttacaattcaagatcttgatctatatccatagattggaatattggtgggccagcaatcctcagacgcctcacttaggacaaatgaggaaactgaggcttggtgaagttacgaaacttgtccaaaatcacacaacttgtaaagggcacagccaagattcagagccaggctgtaaaaattaaaatgaacaaattacggcaaagttttaggagaaagaaggatgtttatgttccagaggccagtcgtccacatcagtggcagacagatgaagaaggcgttcgcaccggaaaatgtagcttcccggttaagtaccttggccatgtagaagttgatgaatcaagaggaatgcacatctgtgaagatgctgtaaaaagattgaaagctgaaaggaagttcttcaaaggcttctttggaaaaactggaaagaaagcagttaaagcagtttctgtgggtctaagcagatggactcagaggttgtggatgaaaaactaaggacctcat

SEQ ID NO: 39 - DNA, 657 bp, Homo sapiens
ctttttgtttgggttttccaatgtagatgtctcagtgaaatgtgcagatatactttgttccttatatggtcaccagtgttaattatggacaaatacattaaaacaagggttcctggcccagcctcccatctaatctctttgatactcttggaatctaagtctgaggagcgatttctgaattagccagtgttgtaccaactttctgttaggaattgtattagaataacctttctttttcagacctgctcagtgagacatcttggggaatgaagtaggaaaatagacatttggtggaaaaacagcaaaatgagaacattaaaaagactcattcaagtatgagtataaagggcatggaaattctggtcctttgagcaaaatgagaagaaaaaattctgctcagcagtattcactgtgttaagattttttgttttttacacgaatggaaaaatgatgtgtaagtggtatagattttaatcagctaacagtcactccagagattttgatcagcaccaattcctatagtagtaagtatttaaaagttaagaaatactactacatttaacattataaagtagagttctggacataactgaaaattagatgtttgcttcaatagaaatttgttcccacttgtattttcaacaaaattatcggaac

SEQ ID NO: 40 - DNA, 1328 bp, Homo sapiens
acaattttaaaataactagcaattaatcacagcatatcaggaaaaagtacacagtgagttctggttagtttttgtaggctcattatggttagggtcgttaagatgtatataagaacctacctatcatgctgtatgtatcactcattccattttcatgttccatgcatactcgggcatcatgctaatatgtatccttttaagcactctcaaggaaacaaaagggccttttatttttataaaggtaaaaaaaattccccaaatattttgcactgaatgtaccaaaggtgaagggacattacaatatgactaacagcaactccatcacttgagaagtataatagaaaatagcttctaaatcaaacttccttcacagtgccgtgtctaccactacaaggactgtgcatctaagtaataattttttaagattcactatatgtgatagtatgatatgcatttatttaaaatgcattagactctcttccatccatcaaatactttacaggatggcatttaatacagatatttcgtatttcccccactgctttttatttgtacagcatcattaaacactaagctcagttaaggagccatcagcaacactgaagagatcagtagtaagaattccattttccctcatcagtgaagacaccacaaattgaaactcagaactatatttctaagcctgcattttcactgatgcataattttcttagtaatattaagagacagtttttctatggcatctccaaaactgcatgacatcactagtcttacttctgcttaattttatgagaaggtattcttcattttaattgcttttgggattactccacatctttgtttatttcttgactaatcagattttcaatagagtgaagttaaattgggggtcataaaagcattggattgacatatggtttgccagcctatgggtttacaggcattgcccaaacatttctttgagatctatatttataagcagccatggaattcctattatgggatgttggcaatcttacattttatagaggtcatatgcatagttttcataggtgttttgtaagaactgattgctctcctgtgagttaagctatgtttactactgggaccctcaagaggaataccacttatgttacactcctgcactaaaggcacgtactgcagtgtgaagaaatgttctgaaaaagggttatagaaatctggaaataagaaaggaagagctctctgtattctataattggaagagaaaaaaagaaaaacttttaactggaaatgttagtttgtacttattgatcatgaatacaagtatatatttaattttgaaaaaa

SEQ ID NO: 41 - DNA, 987 bp, Homo sapiens
aacagagactggcacaggacctcttcattgcaggaagatggtagtgtaggcaggtaacattgagctcttttcaaaaaaggagagctcttcttcaagataaggaagtggtagttatggtggtaacccccggctatcagtccggatggttgccacccctcctgctgtaggatggaagcagccatggagtgggagggaggcgcaataagacacccctccacagagcttggcatcatgggaagctggttctacctcttcctggctcctttgtttaaaggcctggctgggagccttccttttgggtgtctttctcttctccaaccaacagaaaagactgctcttcaaaggtggagggtcttcatgaaacacagctgccaggagcccaggcacagggctgggggcctggaaaaaggagggcacacaggaggagggaggagctggtagggagatgctggctttacctaaggtctcgaaacaaggagggcagaataggcagaggcctctccgtcccaggcccatttttgacagatggcgggacggaaatgcaatagaccagcctgcaagaaagacatgtgttttgatgacaggcagtgtggccgggtggaacaagcacaggccttggaatccaatggactgaatcagaaccctaggcctgccatctgtcagccgggtgacctgggtcaattttagcctctaaaagcctcagtctccttatctgcaaaatgaggcttgtgatacctgttttgaagggttgctgagaaaattaaagataagggtatccaaaatagtctacggccataccaccctgaacgtgcctaatctcgtaagctaagcagggtcaggcctggttagtacctggatggggagagtatggaaaacatacctgcccgcagttggagttggactctgtcttaacagtagcgtggcacacagaaggcactcagtaaatacttgttgaataaatgaagtagcgatttggtgtgaaaaaaa

SEQ ID NO: 42 - DNA, 956 bp, Homo sapiens
cggacggtggggcggacgcgtgggtgcaggagcagggcggctgccgactgccccaaccaaggaaggagcccctgagtccgcctgcgcctccatccatctgtccggccagagccggcatccttgcctgtctaaagccttaactaagactcccgccccgggctggccctgtgcagaccttactcaggggatgtttacctggtgctcgggaagggaggggaaggggccggggagggggcacggcaggcgtgtggcagccacacgcaggcggccagggcggccagggacccaaagcaggatgaccacgcacctccacgccactgcctcccccgaatgcatttggaaccaaagtctaaactgagctcgcagcccccgcgccctccctccgcctcccatcccgcttagcgctctggacagatggacgcaggccctgtccagcccccagtgcgctcgttccggtccccacagactgccccagccaacgagattgctggaaaccaagtcaggccaggtgggcggacaaaagggccaggtgcggcctggggggaacggatgctccgaggactggactgtttttttcacacatcgttgccgcagcggtgggaaggaaaggcagatgtaaatgatgtgttggtttacagggtatatttttgataccttcaatgaattaattcagatgttttacgcaaggaaggacttacccagtattactgctgctgtgcttttgatctctgcttaccgttcaagaggcgtgtgcaggccgacagtcggtgaccccatcactcgcaggaccaagggggcggggactgctggctcacgccccgctgtgtcctccctcccctcccttccttgggcagaatgaattcgatgcgtattctgtggccgccatctgcgcagggtggtggtattctgtcatttacacacgtcgttctaattaaaaagcgaattatactccaaaa

SEQ ID NO: 43 - DNA, 536 bp, Homo sapiens
aaataaacacttccataacattttgttttcgaagtctattaatgcaatcccacttttttccccctagtttctaaatgttaaagagaggggaaaaaaggctcaggatagttttcacctcacagtgttagctgtcttttattttactcttggaaatagagactccattagggttttgacattttgggaacccagttttaccattgtgtcagtaaaacaataagatagtttgagagcatatgatctaaataaagacatttgaagggttagtttgaattctaaaagtaggtaatagccaaatagcattctcatcccttaacagacaaaaacttatttgtcaaaagaattagaaaaggtgaaaatattttttccagatgaaacttgtgccacttccaattgactaatgaaatacaaggagacagactggaaaaagtgggttatgccacctttaaaaccctttctggtaaatattatggtagctaaagggtggtttccccggcacctggacctggacaggtagggttccgtggttaaccagt

SEQ ID NO: 44 - DNA, 1630 bp, Homo sapiens
ggggagggacgagtatggaaccctgaaggtagcaagtccaggcactggcctgaccatccggctccctgggcaccaagtcccaggcaggagcagctgttttccatcccttcccagacaagctctatttttatcacaatgacctttagagaggtctcccaggccagctcaaggtgtcccactatcccctctggagggaagaggcaggaaaattctccccgggtccctgtcatgctactttctccatcccagttcagactgtccaggacatcttatctgcagccataagagaattataaggcagtgatttcccttaggcccaggacttgggcctccagctcatctgttccttctgggcccattcatggcaggttctgggctcaaagctgaactggggagagaagagatacagagctaccatgtgactttacctgattgccctcagtttggggttgcttattgggaaagagagagacaaagagttacttgttacgggaaatatgaaaagcatggccaggatgcatagaggagattctagcaggggacaggattggctcagatgacccctgagggctcttccagtcttgaaatgcattccatgatattaggaagtcgggggtgggtggtggtggtgggctagttgggtttgaatttaggggccgatgagcttgggtacgtgagcagggtgttaagttagggtctgcctgtatttctggtccccttggaaatgtccccttcttcagtgtcagacctcagtcccagtgtccatatcgtgcccagaaaagtagacattatcctgccccatcccttccccagtgcactctgacctagctagtgcctggtgcccagtgacctgggggagcctggctgcaggccctcactggttccctaaaccttggtggctgtgattcaggtccccaggggggactcagggaggaatatggctgagttctgtagtttccagagttggctggtagagccttctagaggttcagaatattagcttcaggatcagctgggggtatggaattggctgaggatcaaacgtatgtaggtgaaaggataccaggatgttgctaaaggtgagggacagtttgggtttgggacttaccagggtgatgttagatctggaacccccaagtgaggctggagggagttaaggtcagtatggaagatagggttgggacagggtgctttggaatgaaagagtgaccttagagggctccttgggcctcaggaatgctcctgctgctgtgaagatgagaaggtgctcttactcagttaatgatgagtgactatatttaccaaagcccctacctgctgctgggtcccttgtagcacaggagactggggctaagggcccctcccagggaagggacaccatcaggcctctggctgaggcagtagcatagaggatccatttctacctgcatttcccagaggactagcaggaggcagccttgagaaaccggcagttcccaagccagcgcctggctgttctctcattgtcactgccctctccccaacctctcctctaacccactagagattgcctgtgtcctgcctcttgcctcttgtagaatgcagctctggccctcaataaatgcttcctgcattcatctgcaaaaaaaaa

SEQ ID NO: 45 - DNA, 169 bp, Homo sapiens
tcttttgcttttagctttttatttttgtattaacaggagtcttattacacataggtctgataaaactggtttatgatcttcagtctgattccagtgctgcataactagataacgtatgaaggaaaaacgacgacgaacaaaaaagtaagtgcttggaagacttagttga

SEQ ID NO: 46 - DNA, 769 bp, Homo sapiens
tgcaggtcatatttactatcggcaataaaaggaagcaaagcagtattaagcagcggtggaatttgtcgctttcactttttataaagtgctacataaaatgtcatatttccaaatttaaaaacataactccagttcttaccatgagaacagcatggtgatcacgaaggatcttcttgaaaaaaacaaaaacaaaaacaaaaaacaatgatctcttctgggtatcacatcaaatgagatacaaaggtgtactaggcaatcttagagatctggcaacttattttatatataaggcatctgtgaccaagagacgttatgaattaaatgtacaaatgtattatgtataaatgtattaaatgcaagcttcatataatgacaccaatgtctctaagttgctcagagatcttgactggctgtggccctggccagctcctttcctgatagtctgattctgccttcatatataggcagctcctgatcatccatgccagtgaatgagaaaacaagcatggaatatataaactttaacattaaaaaatgttttattttgtaataaaatcaaatttcccattgaaaccttcaaaaactttgcagaatgaggttttgatatatgtgtacaagtagtaccttcttagtgcaagaaaacatcattatttctgtctgcctgcctttttgtttttaaaaatgaagactatcattgaaacaagtttgtcttcagtatcaggacatgttgacggagaggaaaggtaggaaagggttagggatagaagcc

SEQ ID NO: 47 - DNA, 2529 bp, Homo sapiens
tttagttcatagtaatgtaaaaccatttgtttaattctaaatcaaatcactttcacaacagtgaaaattagtgactggttaaggtgtgccactgtacatatcatcattttctgactggggtcaggacctggtcctagtccacaagggtggcaggaggagggtggaggctaagaacacagaaaacacacaaaagaaaggaaagctgccttggcagaaggatgaggtggtgagcttgccgagggatggtgggaagggggctccctgttggggccgagccaggagtcccaagtcagctctcctgccttacttagctcctggcagagggtgagtggggacctacgaggttcaaaatcaaatggcatttggccagcctggctttactaacaggttcccagagtgcctctgttggctgagctctcctgggctcactccatttcattgaagagtccaaatgattcattttcctacccacaacttttcattattcttctggaaacccatttctgttgagtccatctgacttaagtcctctctccctccactagttggggccactgcactgaggggggtcccaccaattctctctagagaagagacactccagaggcccctgcaactttgcggatttccagaaggtgataaaaagagcactcttgagtgggtgcccaggaatgtttaaaatctatcaggcacactataaagctggtggtttcttcctaccaagtggattcggcatatgaaccacctactcaatactttatattttgtctgtttaaacactgaactctggtgttgacaggtacaaaggagaagagatggggactgtgaagaggggagggcttccctcatcttcctcaagatctttgtttccataaactatgcagtcataattgagaaaaagcaatagatggggcttcctaccatttgttggttattgctggggttagccaggagcagtgtggatggcaaagtaggagagaggcccagaggaaagcccatctccctccagctttggggtctccagaaagaggctggatttctgggatgaagcctagaaggcagagcaagaactgttccaccaggtgaacagtcctacctgcttggtaccatagtccctcaataagattcagaggaagaagcttatgaaactgaaaatcaaatcaaggtattgggaagaataatttcccctcgattccacaggagggaagaccacacaatatcattgtgctggggctccccaaggccctgccacctggctttacaaatcatcaggggttgcctgcttggcagtcacatgcttccctggttttagcacacatacaaggagttttcagggaactctatcaagccataccaaaatcagggtcacatgtgggtttcccctttccttgcctcttcataaaagacaacttggcttctgaggatggtggtcttttgcatgcagttgggctgacctgacaaagcccccagtttcctgtggcaggttctgggagaggatgcattcaagcttctgcagcctaggggacagggctgcttgttcagttattactgcctcggagctccaaatcccaccaaagtcctgactccaggtctttcctaatgcacagtagtcagtctcagcttcggcagtattctcggctgtatgttctctggcagagagaggcagatgaacatagttttagggagaaagctgatgggaaacctgtgagttaagccacatgtctcaccaggaataatttatgccaggaaaccaggaagtcattcaagttgttctctgaggccaaagacactgagcacagcccagagccaataaaagatctttgagtctctggtgaattcacgaagtgaccccagctttagctactgcaattatgatttttatgggacagcaatttcttgcatctctacagaggaagaagagggggagtgggaggggaaggaaagagaacagagcggcactgggatttgaaaggggaacctctctatctgaggagcccccactggcttcagaagcaacttaccaaggggtatttaaagacatgaaaatttccagaaataccatttggtgcatccctttgtttctgtaatattaaactcaggtgaaattatactctgacagtttctctctttctgcctcttccctctgcagagtcaggacctgcagaactggctgaaacaagatttcatggtgtcacccatgagagatgactcaatgccaaggcctgaagttatagagtgtttacagcggtggcgatattcaggggtcatcgccaactggtctcgagttccaaagctctgatgaagaaacaagactccttgatgtgttactgatcccactgattccaggagtcaagattagccaggaagccaaacaccaggagttggggtggcacgtcaccagtccagagccctgccacggatgtacgcaggagcccagcattaggcaatcaggagccagaacatgatcaccagggccacaaataggaagaggcgtgacaggaactgctcgtccacatacctggggtgtcc

SEQ ID NO: 48 - DNA, 1552 bp, Homo sapiens
tttttttttttttttgatttctgggacaattaagctttatttttcatatatatatatattttcatatatatatatacatacatatataaaggaaacaatttgcaaatttacacacctgacaaaaccatatatacacacatatgtatgcatacacacagacagacacacacacccgaagctctagccaggcccgttttccatccctaagtaccattctctcatttgggcccttctagggttggggccctgagcttggtttgtagaagtttggtgctaatataaccatagctttaatccccatgaaggacagtgtagacctcatctttgtctgctccccgctgcctttcagttttacgtgatccatcaagagggctatgggagccaagtgaacacgggggattgaggctaattcacctgaactcgaaaacagcgcccagcttcctcaccgcaggcacgcgtcttttctttttttttcctcgagacggagtctcgctgtgttgcccaggctggagtgcagtggcacggtctcggctcactgcaagctccacctcctggattcataccattctcctgcttcagccttccgagtagctgggactataggtgccaaccactacgcctagctaatttttttttgtatttttagtagagacagggtttcaccgtgttagccaggatggtctcgtcctgactttgtgatccgcccgcctcggcctcccaaagtgctgggattacaggcgtgagccaccacacctggccccggcacgtatcttttaaggaatgacaccagttcctggcttctgaccaaagaaaaaatgtcacaggagactttgaagaggcagacaggagggtggtggcagcaacactgcagctgcttctggatgctgctggggtgctctccggagcgggtgtgaacagcgcacttcaacatgagcaggcgcctggctccggtgtgtcctcacttcagtggtgcacctggatggtggaagccagcctttggggcaggaaaccagctcagagaggctacccagctcagctgctggcaggagccaggtatttacagccataatgtgtgtaaagaaaaaacacgttctgcaagaaactctcctacccgctcgggagactggggctccttgcttgggatgagcttcactcaacgtggagatggtggtggactggtccctgaaaagcgggccttgcagggccaagtgaggtcctcaggtcctaacccagtggccctctgaaagggggtgtgcaggcgaggggagcaggaggcttctctctagtccctttggaggctttggctgagagaagagtgagcagggagctgggaatggtccaggcagggaagggagctgaagtgattcggggctaatgcctcagatcgatgtatttctctccctggtctcccggagccctcttgtcaccgctgctgccctgcaggaggcccatctcttctgggagcttatctgacttaacttcaactacaagttcgctcttacgagaccgggggtagcgtgatctcctgcttccctgagcgcctgcacggcag

SEQ ID NO: 49 - DNA, 921 bp, Homo sapiens
ctgtggtcccagctactcaggaggctgaggcgggaggattgcttgagcccaggagttggatgttgcagtgagccaagatcgcaccattgccctccactctgggccacggagcaataccctgtctcagaaaacaaacaacaaaaagcagaaacgctgaaggggtcggtttacgggaaaaccgcctgtcagaacacttggctactcctaccccagatcagtggacctgggaatgagggttggtcccgggaggcttttctccaagctgttgccaccagacccgccatgggaaccctggccacagaagcctcccggggagtgagccagagcctggaccgctgtgctgatgtgtctggggtggagggagggtggggagtgtgcaagggtgtgtgtgtgcccggggggtgttcatgggcaagcatgtgcgtgcctgtgtgtgtgcgtgcccctcccctgcagccgtcggtggtatctccctccagccccttcgccaccttctgagcattgtctgtccacgtgagactgcccagagacagcagagctccacgtggttttaaggggagacctttccctggacctgggggtctcgccgtatctcatgaccaggtgctaaatgacccgacatgcatcacctgcctttcgatgaccaacctccctgtccccgtcccgctgacctgcccccgtggcgtctcacggtgatgcctgctcctgacattggtgttcactgtagcaaactacattctggatgggaattttcatgtacatgtgtggcatgtggaaaatttcaaataaaatggacttgatttagaaagccaaaaagctgtgtggtccttccagcacggatactttgacctcttgcctacaaccccttccttgggtccgaggctggtagctttgttcacttcagatggttgggggcgggtg

SEQ ID NO: 50 - DNA, 338 bp, Homo sapiens
atgatctatctagatgccctaccgtaaaatcaaaacacaaaaccctactgactcattccctcccttccagatattaccccatttctctacttcccattgtagccaaactttccaaaaattcatgttctgtcttcatttcctcatgttcaacccaccctgtcttagctaccacccctcagtaacgacctagcctgggtagaaacaaatgtcagcatgataccatactcaatgatccttcgtcactgttgtcattgtcatcattccatggccttactttccctctcagcgccatttgctacagtaagaaactttctttcttgaattcttggttctcttgg

SEQ ID NO: 51 - DNA, 1191 bp, Homo sapiens
ctagcaagcaggtaaacgagctttgtacaaacacacacagaccaacacatccggggatggctgtgtgttgctagagcagaggctgattaaacactcagtgtgttggctctctgtgccactcctggaaaataatgaattgggtaaggaacagttaataagaaaatgtgccttgctaactgtgcacattacaacaaagagctggcagctcctgaaggaaaagggcttgtgccgctgccgttcaaacttgtcagtcaactcatgccagcagcctcagcgtctgcctccccagcacaccctcattacatgtgtctgtctggcctgatctgtgcatctgctcggagacgctcctgacaagtcgggaatttctctatttctccactggtgcaaagagcggatttctccctgcttctcttctgtcacccccgctcctctcccccaggaggctccttgatttatggtagctttggacttgcttccccgtctgactgtccttgacttctagaatggaagaagctgagctggtgaagggaagactccaggccatcacagataaaagaaaaatacaggaagaaatctcacagaagcgtctgaaaatagaggaagacaaactaaagcaccagcatttgaagaaaaaggccttgagggagaaatggcttctagatggaatcagcagcggaaaagaacaggaagagatgaagaagcaaaatcaacaagaccagcaccagatccaggttctagaacaaagtatcctcaggcttgagaaagagatccaagatcttgaaaaagctgaactgcaaatctcaacgaaggaagaggccattttaaagaaactaaagtcaattgagcggacaacagaagacattataagatctgtgaaagtggaaagagaagaaagagcagaagagtcaattgaggacatctatgctaatatccctgaccttccaaagtcctacataccttctaggttaaggaaggagataaatgaagaaaaagaagatgatgaacaaaataggaaagctttatatgccatggaaattaaagttgaaaaagacttgaagactggagaaagtacagttctgtcttccaatacctctggccatcagatgactttaaaaggtacaggagtaaaagtttaagatgatgggcaaaagtccagtgtattcagtaaagtgctaatcacaagttggaggt

SEQ ID NO: 52 - DNA, 1200 bp, Homo sapiens
aacagggactctcactctatcaaccccaggctggagtccggtgcgcccaccctggctccctgcaacctccgcctcccaggctcaagcaactctcctgcctcagtcgctctagtagctgggactacaggcacacaccaccatgcccagccaatttttgcattttttgtagagacagggtttcgccttctgtccaggccggcatcatatactttaaatcatgcccagatgactttaatacctaatacaatatatcaggttggtttaaaaataattgcttttttattatttttgcatttttgcaccaaccttaatgctatgtaaatagttgttatactgttgcttaacaacagtatgacaattttggctttttctttgtattattttgtattttttttttttattgtgtggtcttttttttttttctcagtgttttcaattcctccttggttgaatccatggatgcaaaacccacagatatgaagggctggctatatatgcattgatgattgtcctattatattagttataaagtgtcatttaatatgtagtgaaagttatggtacagtggaaagagtagttgaaaacataaacatttggacctttcaagaaaggtagcttggtgaagtttttcaccttcaaactatgtcccagtcagggctctgctactaattagctataatctttgcacaaattacatcacctttgagtctcagttgcctcacctgtaaaatgaaagaactggatactctctaaggtcacttccagccctgtcattctataactctgttatgctgaggaagaaattcacattgtgttaactgtatgagtcaaactgaaaatgattattaaagtgggaaaaagccaattgcttctcttagaaagctcaactaaatttgagaagaataatcttttcaattttttaagaatttaaatatttttaagggtttgacctatttatttagagatggggtctcactctgtcacccagactggagtacagtggcacaatcatagctcactgctgcctcaaattcatgggctcaagtgatcctcctgcctctgcctccagagtagctgcgactatgggcatgtgccaccacgcctggctaacatttgtattgacctatttatttattgtgatttatatctttttttttttttcttttttttttttttacaaaatcagaaatacttattttg

SEQ ID NO: 53 - DNA, 989 bp, Homo sapiens
aagccaccactcaaaacttcctatacattttcacagcagagacaagtgaacatttatttttatgcctttcttcctatgtgtatttcaagtctttttcaaaacaaggccccaggactctccgattcaattagtccttgggctggtcgactgtgcaggagtccagggagcctctacaaatgcagagtgactctttaccaacataaaccctagatacatgcaaaaagcaggacccttcctccaggaatgtgccatttcagatgcacagcacccatgcagaaaagctggaattttccttggaaccgactgtgatagaggtgcttacatgaacattgctactgtctttctttttttttgagacaggtttcgcttgtgcccaggctgagtgcaatgcgtgatctcactcactgcaattccacctccaggttcaagcattctcctgctcagcctcctagtagctgggttacaggcactgccaccatgccggctaattttgtatttttgtagagatggatttctccatttggtcaggcggtctcgaaccccaacctcagtgatctgccacctcagcctcctaagtgttggattacaggatgagccacccgaccggccactactgtctttctttgacccttccagtttcgaagataaagaggaaataatttctctgaagtacttgataaaatttccaaacaaaacacatgtccacttcactgataaaaaatttaccgcagtttggcacctaagagtatgacaacagcaataaaaagtaatttcaaagagttaagatttcttcagcaaaatagatgattcacatcttcaagtcctttttgaaatcagttattaatattattctttcctcatttccatctgaatgactgcagcaatagtttttttttttttttttttttttttgcgagatggaatctcgctctgtcgcccagcgggagtgcactggcgcaagcccggctcaccgcaatctctgccacccg

SEQ ID NO: 54 - DNA, 250 bp, Homo sapiens
catttccccattggtcctgatgttgaagatttagttaaagaggctgtaagtcaggttcgagcagaggctactacaagaagtagggaatcaagtccctcacatgggctattaaaactaggtagtggtggagtagtgaaaaagaaatctgagcaacttcataacgtaactgcctttcagggaaaagggcattctttaggaactgcatctggtaacccacaccttgatccaagagctagggaaacttcagttg

SEQ ID NO: 55 - DNA, 2270 bp, Homo sapiens
gcgcccccgagcagcgcccgcgccctccgcgccttctccgccgggacctcgagcgaaagaggcccgcgcgccgcccagccctcgcctccctgcccaccgggcacaccgcgccgccaccccgaccccgctgcgcacggcctgtccgctgcacaccagcttgttggcgtcttcgtcgccgcgctcgccccgggctactcctgcgcgccacaatgagctcccgcatcgccagggcgctcgccttagtcgtcacccttctccacttgaccaggctggcgctctccacctgccccgctgcctgccactgccccctggaggcgcccaagtgcgcgccgggagtcgggctggtccgggacggctgcggctgctgtaaggtctgcgccaagcagctcaacgaggactgcagcaaaacgcagccctgcgaccacaccaaggggctggaatgcaacttcggcgccaagtccaccgctctgaaggggatctgcagagctcagtcagagggcagaccctgtgaatataactccagaatctaccaaaacggggaaagtttccagcccaactgtaaacatcagtgcacatgtattgatggcgccgtgggctgcattcctctgtgtccccaagaactatctctccccaacttgggctgtcccaaccctcggctggtcaaagttaccgggcagtgctgcgaggagtgggtctgtgacgaggatagtatcaaggaccccatggaggaccaggacggcctccttggcaaggagctgggattcgatgcctccgaggtggagttgacgagaaacaatgaattgattgcagttggaaaaggcagctcactgaagcggctccctgtttttggaatggagcctcgcatcctatacaaccctttacaaggccagaaatgtattgttcaaacaacttcatggtcccagtgctcaaagacctgtggaactggtatctccacacgagttaccaatgacaaccctgagtgccgccttgtgaaagaaacccggatttgtgaggtgcggccttgtggacagccagtgtacagcagcctgaaaaagggcaagaaatgcagcaagaccaagaaatcccccgaaccagtcaggtttacttacgctggatgtttgagtgtgaagaaataccggcccaagtactgcggttcctgcgtggacggccgatgctgcacgccccagctgaccaggactgtgaagatgcggttccgctgcgaagatggggagacattttccaagaacgtcatgatgatccagtcctgcaaatgcaactacaactgcccgcatgccaatgaagcagcgtttcccttctacaggctgttcaatgacattcacaaatttagggactaaatgctacctgggtttccagggcacacctagacaaacaagggagaagagtgtcagaatcagaatcatggagaaaatgggcgggggtggtgtgggtgatgggactcattgtagaaaggaagccttgctcattcttgaggagcattaaggtatttcgaaactgccaagggtgctggtgcggatggacactaatgcagccacgattggagaatactttgcttcatagtattggagcacatgttactgcttcattttggagcttgtggagttgatgactttctgttttctgtttgtaaattatttgctaagcatattttctctaggcttttttccttttggggttctacagtcgtaaaagagataataagattagttggacagtttaaagcttttattcgtcctttgacaaaagtaaatgggagggcattccatcccttcctgaagggggacactccatgagtgtctgtgagaggcagctatctgcactctaaactgcaaacagaaatcaggtgttttaagactgaatgttttatttatcaaaatgtagcttttggggagggaggggaaatgtaatactggaataatttgtaaatgattttaattttatattcagtgaaaagattttatttatggaattaaccatttaataaagaaatatttacctaatatctgagtgtatgccattcggtatttttagaggtgctccaaagtcattaggaacaacctagctcacgtactcaattattcaaacaggacttattgggatacagcagtgaattaagctattaaaataagataatgattgcttttataccttcagtagagaaaagtctttgcatataaagtaatgtttaaaaaacatgtattgaacacgacattgtatgaagcacaataaagattctgaagctaaaaaaaaaa

SEQ ID NO: 56 - DNA, 1636 bp, Homo sapiens
cttgaatgaagctgacaccaagaaccgcgggaagagcttgggcccaaagcaggaaagggaagcgctcgagttggaaaggaaccgctgctgctggccgaactcaagcccgggcgcccccaccagtttgattggaagtccagctgtgaaacctggagcgtcgccttctccccagatggctcctggtttgcttggtctcaaggacactgcatcgtcaaactgatcccctggccgttggaggagcagttcatccctaaagggtttgaagccaaaagccgaagtagcaaaaatgagacgaaagggcggggcagcccaaaagagaagacgctggactgtggtcagattgtctgggggctggccttcagcccgtggccttccccacccagcaggaagctctgggcacgccaccacccccaagtgcccgatgtctcttgcctggttcttgctacgggactcaacgatgggcagatcaagatctgggaggtgcagacagggctcctgcttttgaatctttccggccaccaagatgtcgtgagagatctgagcttcacacccagtggcagtttgattttggtctccgcgtcacgggataagactcttcgcatctgggacctgaataaacacggtaaacagattcaagtgttatcgggccacctgcagtgggtttactgctgttccatctccccagactgcagcatgctgtgctctgcagctggagagaagtcggtctttctatggagcatgaggtcctacacgttaattcggaagctagagggccatcaaagcagtgttgtctcttgtgacttctcccccgactctgccctgcttgtcacggcttcttacgataccaatgtgattatgtgggacccctacaccggcgaaaggctgaggtcactccaccacacccaggttgaccccgccatggatgacagtgacgtccacattagctcactgagatctgtgtgcttctctccagaaggcttgtaccttgccacggtggcagatgacagactcctcaggatctgggccctggaactgaaaactcccattgcatttgctcctatgaccaatgggctttgctgcacattttttccacatggtggagtcattgccacagggacaagagatggccacgtccagttctggacagctcctagggtcctgtcctcactgaagcacttatgccggaaagcccttcgaagtttcctaacaacttaccaagtcctagcactgccaatccccaagaaaatgaaagagttcctcacatacaggactttttaagcaacaccacatcttgtgcttctttgtagcagggtaaatcgtcctgtcaaagggagttgctggaataatgggccaaacatctggtcttgcattgaaatagcatttctttgggattgtgaatagaatgtagcaaaaccagattccagtgtacataaaagaatttttttgtctttaaatagatacaaatgtctatcaactttaatcaagttgtaacttatattgaagacaatttgatacataataaaaaattatgacaatgtcctgggaaaaaaaaaatgtagaaagatggtgaagggtgggatggatgaggagcgtggtgacgggggcctgcagcgggttggggaccctgtgctgcgtt

SEQ ID NO: 57 - DNA, 460 bp, Homo sapiens
ccatgtgtgtatgagagagagagagattgggagggagagggagctcactagcgcatatgtgcctccagggggctgcagatgtgtctgagggtgagcctggtgaaagagaagacaaaagaatggaatgagctaaagcagccgcctggggtgggaggccgagcccatttgtatgcagcagggggcaggagcccagcaagggagcctccattcccaggactctggagggagctgagaccatccatgcccgcagagccctccctcacactccatcctgtccagccctaattgtgcaggtggggaaactgaggctgggaagtcacatagcaagtgactggcagagctgggactggaacccaaccagcctcctagaccacggttcttcccatcaatggaatgctagagactccagccaggtgggtaccgagctcgaattcgtaatcatggtcatagctgtttcctg

SEQ ID NO: 58 - DNA, 1049 bp, Homo sapiens
atctgatcaagaatacctgccctggtcactctgcggatgtttctgtccacttgttcacattgaggaccaagatatccttttttacagaggcacttgttcggtctaacacagacacctccatgacgacatgctggctcacattttgcagttctgcagaagtccccctcccagcctggactacagcagcactttcccgtgggggtgcagtagccgtttcgacagagcctggagcactctgaagtcagtgtctgtgcaggttgtaccgtggctctgcattcctcaggcattaaaggtcttttgggatctacaattttgtagagttttccattgtgagtctgggtcatacttttactgcttgataaaatgtaaacttcacctagttcatcttctccaaatcccaagatgtgaccggaaaagtagcctctacaggacccactagtgccgacacagagtggtttttcttgccactgctttgtcacaggactttgctggagagttaggaaattcccattacgatctccaaacacgtagcttccatacaatctttctgactggcagccccggtatacaaatccaccaaccaaaggaccattactgaatggcttgaattctaaaagtgatggctcactttcataatctttcccctttattatctgtagaattctggctgatgatctgttttttccattggagtctgaacacagtatcgttaaattgatgtttatatcagtgggatgtctatccacagcacatctgcctggatcgtggagcccatgagcaaacacttcggggggctggttggtgctgttgaagtgtgggttgctccttggtatggaataaggcacgttgcacatgtctgtgtccacatccagccgtagcactgagcctgtgaaatcacttaacccatccatttcttccatatcatccagtgtaatcatcccatcaccaagaatgatgtacaaaaacccgtcagggccaaagagcagttgccctcccagatgctttctgtggagttctgcaacttcaagaaagactctggctgttctcaa

SEQ ID NO: 59 - DNA, 747 bp, Homo sapiens
tttttcaaatcacatatggcttctttgaccccatcaaataactttattcacacaaacgtcccttaatttacaaagcctcagtcattcatacacattaggggatccacagtgttcaaggaacttaaatataatgtatcataccaacccaagtaaaccaagtacaaaaaatattcatataaagttgttcacacgtaggtcctagattaccagcttctgtgcaaaaaaaggaaatgaagaaaaatagatttattaactagtattggaaactaactttgtgcctggcttaaaacctccctcacgctcgtctgtcccacacaaatgtttaagaagtcactgcaatgtactccccggctctgatgaaaagaagcccctggcacaaaagattccagtgcccctgaagaggctcccttcctcctgtgggctctcctagaaaaccagcgggacggcctccctgctgataccgtctataaccttagggggccctcgggcaggcaacggcagtggactcatctcggtgatggctgtagatgctaacactggccaattcaatgccacacctactggttaccctttgagggcatttctccagacagaagccccttgaagcctaggtagggcaggatcagagatacacccgtgtttgtctcgaagggctccacagcccagtacgacatgcttgcagaagtagtatctctggacttctgcctccagtcgaccggccgcgaatttagtagtaatagcggccgc

SEQ ID NO: 60 - protein, 1036 aa, Homo sapiens
MYLVAGDRGLAGCGHLLVSLLGLLLLPARSGTRALVCLPCDESKCEEPRNRPGSIVQGVCGCCYTCASQGNESCGGTFGIYGTCDRGLRCVIRPPLNGDSLTEYEAGVCEDENWTDDQLLGFKPCNENLIAGCNIINGKCECNTIRTCSNPFEFPSQDMCLSALKRIEEEKPDCSKARCEVQFSPRCPEDSVLIEGYAPPGECCPLPSRCVCNPAGCLRKVCQPGNLNILVSKASGKPGECCDLYECKPVFGVDCRTVECPTVQQTACPPDSYETQVRLTADGCCTLPTRCECLSGLCGFPVCEVGSTPRIVSRGDGTPGKCCDVFECVNDTKPACVFNNVEYYDGDMFRMDNCRFCRCQGGVAICFTAQCGEINCERYYVPEGECCPVCEDPVYPFNNPAGCYANGLILAHGDRWREDDCTFCQCVNGERHCVATVCGQTCTNPVKVPGECCPVCEEPTIITVDPPACGELSNCTLTRKDCINGFKRDHNGCRTCQCINTQELCSERKQGCTLNCPFGFLTDAQNCEICECRPRPKKCRPIICDKYCPLGLLKNKHGCDICRCKKCPELSCSKICPLGFQQDSHGCLICKCREASASAGPPILSGTCLTVDGHHHKNEESWHDGCRECYCLNGREMCALITCPVPACGNPTIHPGQCCPSCADDFVVQKPELSTPSICHAPGGEYFVEGETWNIDSCTQCTCHSGRVLCETEVCPPLLCQNPSRTQDSCCPQCTDQPFRPSLSRNNSVPNYCKNDEGDIFLAAESWKPDVCTSCICIDSVISCFSESCPSVSCERPVLRKGQCCPYCIKDTIPKKVVCHFSGKAYADEERWDLDSCTHCYCLQGQTLCSTVSCPPLPCVEPINVEGSCCPMCPEMYVPEPTNIPIEKTNHRGEVDLEVPLWPTPSENDIVHLPRDMGHLQVDYRDNRLHPSEDSSLDSIASVVVPIIICLSIIIAFLFINQKKQWIPLLCWYRTPTKPSSLNNQLVSVDCKKGTRVQVDSSQRMLRIAEPDARFSGFYSMQKQNHLQADNFYQTV

(19) United States
(12) Patent Application Publication
(10) Pub. No.: US 2004/0147449 A1
(43) Pub. Date: Jul. 29, 2004

(54) Combinations and compositions which interfere with VEGF/ VEGF and angiopoietin/ Tie receptor function and their use (II)

(76) Inventors: Gerhard Siemeister, Berlin (DE); Martin Haberey, Berlin (DE); Karl-Heinz Thierauch, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/796,174

(22) Filed: Mar. 10, 2004

Related U.S. Application Data
(63) Continuation of application No. 09/887,527, filed on Jun. 25, 2001, now abandoned.

(30) Foreign Application Priority Data
Jun. 23, 2000 (EP) 00250194.8
Jun. 28, 2000 (EP) 00250214.4

(51) Int. Cl. A61K 38/17

(52) U.S. Cl. 514/12

(57) ABSTRACT

The present invention describes the combination of substances interfering with the biological activity of Vascular Endothelial Growth Factor (VEGF)/VEGF receptor systems (compound I) and substances interfering with the biological function of Angiopoietin/Tie receptor systems (compound II) for inhibition of vascularization and for cancer treatment.

The present invention provides the combination of substances interfering with the biological activity of Vascular Endothelial Growth Factor (VEGF)/VEGF receptor systems (compound I) and substances interfering with the biological function of Angiopoietin/Tie receptor systems (compound II) for inhibition of vascularization and for cancer treatment.

Protein ligands and receptor tyrosine kinases that specifically regulate endothelial cell function are substantially involved in physiological as well as in disease-related angiogenesis. These ligand/receptor systems include the Vascular Endothelial Growth Factor (VEGF) and the Angiopoietin (Ang) families, and their receptors, the VEGF receptor family and the tyrosine kinase with immunoglobulin-like and epidermal growth factor homology domains (Tie) family. The members of the two families of receptor tyrosine kinases are expressed primarily on endothelial cells. The VEGF receptor family includes Flt1 (VEGF-R1), Flk1/KDR (VEGF-R2), and Flt4 (VEGF-R3). These receptors are recognized by members of the VEGF-related growth factors in that the ligands of Flt1 are VEGF and placenta growth factor (PIGF), whereas Flk1/KDR binds VEGF, VEGF-C and VEGF-D, and the ligands of Flt4 are VEGF-C and VEGF-D (Nicosia, Am. J. Pathol. 153, 11-16, 1998). The second family of endothelial cell specific receptor tyrosine kinases is represented by Tie1 and Tie2 (also kown as Tek). Whereas Tie1 remains an orphan receptor, three secreted glycoprotein ligands of Tie2, Ang1, Ang2, and Ang3/Ang4 have been discovered (Davis et al., Cell 87, 1161-1169, 1996; Maisonpierre et al., Science 277, 55-60, 1997; Valenzuela et al, Proc. Natl. Acad. Sci. USA 96, 1904-1909, 1999; patents: U.S. Pat. No. 5,521,073; U.S. Pat. No. 5,650,490; U.S. Pat. No. 5,814,464).

The pivotal role of VEGF and of its receptors during vascular development was exemplified in studies on targeted gene inactivation. Even the heterozygous disruption of the VEGF gene resulted in fatal deficiencies in vascularization (Carmeliet et al., Nature 380, 435-439, 1996; Ferrara et al., Nature 380, 439-442, 1996). Mice carrying homozygous disruptions in either Flt1 or Flk1/KDR gene die in mid-gestation of acute vascular defects. However, the phenotypes are distinct in that Flk1/KDR knock-out mice lack both endothelial cells and a developing hematopoietic system (Shalaby et al. Nature 376, 62-66, 1995), whereas Flt1 deficient mice have normal hematopoietic progenitors and endothelial cells, which fail to assemble into functional vessels (Fong et al., 376, 66-70, 1995). Disruption of the Flt4 gene, whose extensive embryonic expression becomes restricted to lymphatic vessels in adults, revealed an essential role of Flt4 for the remodeling and maturation of the primary vascular networks into larger blood vessels during early development of the cardiovascular system (Dumont et al., Science 282, 946-949, 1998). Consistent with the lymphatic expression of Flt4 in adults overexpression of VEGF-C in the skin of transgenic mice resulted in lymphatic, but not vascular, endothelial proliferation and vessel enlargement (Jeltsch et al., Science 276, 1423-1425, 1997). Moreover, VEGF-C was reported to induce neovascularization in mouse cornea and chicken embryo chorioallantoic membrane models of angiogenesis (Cao et al., Proc. Natl. Acad. Sci. USA 95, 14389-14394, 1998).

The second class of endothelial cell specific receptor tyrosine kinases has also been found to be critically involved in the formation and integrity of vasculature. Mice deficient in Tie1 die of edema and hemorrhage resulting from poor structural integrity of endothelial cells of the microvasculature (Sato et al., Nature 376, 70-74, 1995; Rodewald & Sato, Oncogene 12, 397-404, 1996). The Tie2 knock-out phenotype is characterized by immature vessels lacking branching networks and lacking periendothelial support cells (Sato et al., Nature 376, 70-74, 1995; Dumont et al., Genes Dev. 8, 1897-1909, 1994). Targeted inactivation of the Tie2 ligand Ang1, as well as overexpression of Ang2, an inhibitory ligand, resulted in phenotypes similar to the Tie2 knock out (Maisonpierre et al., Science 277, 55-60, 1997; Suri et al., cell 87, 1171-1180). Conversely, increased vascularization was observed upon transgenic overexpression of Ang1 (Suri et al., Science 282, 468-471, 1998; Thurstonen et al., Science 286, 2511-2514, 1999).

The results from angiogenic growth factor expression studies in corpus luteum development (Maisonpierre et al., Science 277, 55-60, 1997; Goede et al. Lab. Invest. 78, 1385-1394, 1998), studies on blood vessel maturation in the retina (Alon et al., Nature Med. 1, 1024-1028, 1995; Benjamin et al, Development 125, 1591-1598, 1998), and gene targeting and transgenic experiments on Tie2, Ang1, and Ang2, suggest a fundamental role of the Angiopoietin/Tie receptor system in mediating interactions between endothelial cells and surrounding pericytes or smooth muscle cells. Ang1, which is expressed by the periendothelial cells and seems to be expressed constitutively in the adult, is thought to stabilize existing mature vessels. Ang2, the natural antagonist of Ang1 which is expressed by endothelial cells at sites of vessel sprouting, seems to mediate loosening of endothelial-periendothelial cell contacts to allow vascular remodeling and sprouting in cooperation With angiogenesis initiators such as VEGF, or vessel regression in the absence of VEGF (Hanahan, Science 277, 48-50, 1997).

In pathological settings associated with aberrant neovascularization elevated expression of angiogenic growth factors and of their receptors has been observed. Most solid tumors express high levels of VEGF and the VEGF receptors appear predominantly in endothelial cells of vessels surrounding or penetrating the malignant tissue (Plate et al., Cancer Res. 53, 5822-5827, 1993). Interference with the VEGF/VEGF receptor system by means of VEGF-neutralizing antibodies (Kim et al., Nature 362, 841-844, 1993), retroviral expression of dominant negative VEGF receptor variants (Millauer et al., Nature 367, 576-579, 1994), recombinant VEGF-neutralizing receptor variants (Goldman et al., Proc. Natl. Acad. Sci. USA 95, 8795-8800, 1998), or small molecule inhibitors of VEGF receptor tyrosine kinase (Fong et al., Cancer Res. 59, 99-106, 1999; Wedge et al., Cancer Res. 60, 970-975, 2000; Wood et al. Cancer Res. 60, 2178-2189, 2000), or targeting cytotoxic agents via the VEGF/VEGF receptor system (Arora et al., Cancer Res. 59, 183-188, 1999; EP 0696456A2) resulted in reduced tumor growth and tumor vascularization. However, although many tumors were inhibited by interference with the VEGF/VEGF receptor system, others were unaffected (Millauer et al., Cancer Res. 56, 1615-1620, 1996). Human tumors as well as experimental tumor xenografts contain a large number of immature blood vessels that have not yet recruited periendothelial cells. The fraction of immature vessels is in the range of 40% in slow growing prostate cancer and 90% in fast growing glioblastoma. A selective obliteration of immature tumor vessels was observed upon withdrawal of VEGF by means of downregulation of VEGF transgene expression in a C6 glioblastoma xenograft model. This result is in accordance with a function of VEGF as endothelial cell survival factor. Similarly, in human prostate cancer shutting off VEGF expression as a consequence of androgen-ablation therapy led to selective apoptotic death of endothelial cells in vessels lacking periendothelial cell coverage. In contrast, the fraction of vessels which resisted VEGF withdrawal showed periendothelial cell coverage (Benjamin et al., J. Clin. Invest. 103, 159-165, 1999).

The observation of elevated expression of Tie receptors in the endothelium of metastatic melanomas (Kaipainen et al., Cancer Res. 54, 6571-6577, 1994), in breast carcinomas (Salven et al., Br. J. Cancer 74, 69-72, 1996), and in tumor xenografts grown in the presence of dominant-negative VEGF receptors (Millauer et al., Cancer Res. 56, 1615-1620, 1996), as well as elevated expression of Flt4 receptors in the endothelium of lymphatic vessels surrounding lymphomas and breast carcinomas (Jussila et al., Cancer Res. 58, 1599-1604, 1998), and of VEGF-C in various human tumor samples (Salvén et al., Am. J. Pathol. 153, 103-108, 1998), suggested these endothelium-specific growth factors and receptors as candidate alternative pathways driving tumor neovascularization. The high upregulation of Ang2 expression already in early tumors has been interpreted in terms of a host defense mechanism against initial cooption of existing blood vessels by the developing tumor. In the absence of VEGF, the coopted vessels undergo regression leading to necrosis within the center of the tumor. Contrarily, hypoxic upregulation of VEGF expression in cooperation with elevated Ang2 expression rescues and supports tumor vascularization and tumor growth at the tumor margin (Holash et al., Science 284, 1994-1998, 1999; Holash et al., Oncogene 18, 5356-5362, 1999).

Interference with Tie2 receptor function by means of Angiopoietin-neutralizing Tie2 variants consisting of the extracellular ligand-binding domain has been shown to result in inhibition of growth and vascularization of experimental tumors (Lin et al., J. Clin. Invest. 103, 159-165, 1999; Lin et al. Proc. Natl. Acad. Sci. USA 95, 8829-8834, 1998; Siemeister et al., Cancer Res. 59, 3185-3191, 1999). Comparing the effects of interference with the endothelium-specific receptor tyrosine kinase pathways by means of paracrine expression of the respective extracellular receptor domains on the same cellular background demonstrated inhibition of tumor growth upon blockade of the VEGF receptor system and of the Tie2 receptor system, respectively (Siemeister et al., Cancer Res. 59, 3185-3191, 1999).

It is known that the inhibition of the VEGF/VEGR receptor system by various methods resulted only in slowing down growth of most experimental tumors (Millauer et al., Nature 367, 576-579, 1994; Kim et al., Nature 362, 841-844, 1993; Millauer et al., Cancer Res. 56, 1615-1620, 1996; Goldman et al., Proc. Natl. Acad. Sci. USA 95, 8795-8800, 1998; Fong et al., Cancer Res. 59, 99-106, 1999; Wedge et al., Cancer Res. 60, 970-975, 2000; Wood et al. Cancer Res. 60, 2178-2189, 2000; Siemeister et al., Cancer Res. 59, 3185-3191, 1999). Even by escalation of therapeutic doses a plateau level of therapeutic efficacy was achieved (Kim et al., Nature 362, 841-844, 1993; Wood et al. Cancer Res. 60, 2178-2189, 2000). Similar results were observed upon interference with the Angiopoietin/Tie2 receptor system (Lin et al., J. Clin. Invest. 103, 159-165, 1999; Lin et al., Proc. Natl. Acad. Sci. USA 95, 8829-8834, 1998; Siemeister et al., Cancer Res. 59, 3185-3191, 1999).

However, there is a high demand for methods that enhance the therapeutic efficacy of anti-angiogenous compounds.

Searching for methods that enhance the therapeutic efficacy of anti-angiogenic compounds, superior anti-tumor effects were observed unexpectedly upon combination of inhibition of VEGF/VEGF receptor systems and interference with biological function of Angiopoietin/Tie receptor systems. The mode of action underlying the superior effects observed may be that interference biological function of Angiopoietin/Tie receptor systems destabilizes endothelial cell-periendothelial cell interaction of existing mature tumor vessels and thereby sensitizes the endothelium to compounds directed against VEGF/VEGF receptor systems.

Based on this unexpected finding the present invention provides the combination of functional interference with VEGF/VEGF receptor systems and with Angiopoietin/Tie receptor systems for inhibition of vascularization and of tumor growth.

The pharmaceutical composition consists of two components: compound I inhibits the biological activity of one or several of the VEGF/VEGF receptor systems or consists of cytotoxic agents which are targeted to the endothelium via recognition of VEGF/VEGF receptor systems. Compound II interferes with the biological function of one or several of Angiopoietin/Tie receptor systems or consists of cytotoxic agents which are targeted to the endothelium via recognition of Angiopoietin/Tie receptor systems. Alternatively, compound I inhibits the biological activity of one or several of the VEGF/VEGF receptor systems or of the Angiopoietin/Tie receptor systems and coumpound II consists of cytotoxic agents which are targeted to the endothelium via recognition of one or several of the VEGF/VEGF receptor systems or of the Angiopoietin/Tie receptor systems. Targeting or modulation of the biological activities of VEGF/VEGF receptor systems and of Angiopietin/Tie receptor systems can be performed by

(a) compounds which inhibit receptor tyrosine kinase activity,

(b) compounds which inhibit ligand binding to receptors,

(c) compounds which inhibit activation of intracellular signal pathways of the receptors,

(d) compounds which inhibit or activate expression of a ligand or of a receptor of the VEGF or Tie receptor system,

(e) delivery systems, such as antibodies, ligands, high-affinity binding oligonucleotides or oligopeptides, or liposomes, which target oytotoxic agents or coagulation-inducing agents to the endothelium via recognition of VEGF/VEGF receptor or Angiopoietin/Tie receptor systems,

(f) delivery systems, such as antibodies, ligands, high-affinity binding oligonucleotides or oligopeptides, or liposomes, which are targeted to the endothelium and induce necrosis or apoptosis.

A compound comprised by compositions of the present invention can be a small molecular weight substance, an oligonucleotide, an oligopeptide, a recombinant protein, an antibody, or conjugates or fusionproteins thereof. An example of an inhibitor is a small molecular weight molecule which inactivates a receptor tyrosine kinase by binding to and occupying the catalytic site such that the biological activity of the receptor is decreased. Kinase inhibitors are known in the art (Sugen: SU5416, SU6668; Fong et al. (1999), Cancer Res. 59, 99-106; Vajkoczy et al., Proc. Am. Associ. Cancer Res. San Francisco (2000), Abstract ID 3612; Zeneca: ZD4190, ZD6474; Wedge et al. (2000), Cancer Res. 60, 970-975; Parke-Davis PD0173073, PD0173074; Johnson et al., Proc. Am. Associ. Cancer Res., San Franzisco (2000), Abstract ID 3614; Dimitroff et al. (1999), Invest. New Drugs 17, 121-135). An example of an antagonist is a recombinant protein or an antibody which binds to a ligand such that activation of the receptor by the ligand is prevented. Another example of an antagonist is an antibody which binds to the receptor such that activation of the receptor is prevented. An example of an expression modulator is an antisense RNA or ribozyme which controls expression of a ligand or a receptor. An example of a targeted cytotoxic agent is a fusion protein of a ligand with a bacterial or plant toxin such as Pseudomonas exotoxin A, Diphtheria toxin, or Ricin A. An example of a targeted coagulation-inducing agent is a conjugate of a single chain antibody and tissue factor. Ligand-binding inhibitors such as neutralizing antibodies which are known in the art are described by Genentech (rhuMAbVEGF) and by Presta et al. (1997), Cancer Res. 57, 4593-4599. Ligand-binding receptor domaines are described by Kendall & Thomas (1993), Proc. Natl. Acad. Sci., U.S.A.90, 10705-10709; by Goldman et al. (1998) Proc. Natl. Acad. Sci., U.S.A.95, 8795-8800 and by Lin et al. (1997), J. Clin. Invest. 100, 2072-2078. Further, dominant negative receptors have been described by Millauer et al. (1994), Nature 367, 567-579.

Receptor blocking antibodies have been described by Imclone (cp1C11, U.S. Pat. No. 5,874,542). Further known are antagonistic ligand mutants (Siemeister et al. (1998), Proc. Natl. Acad. Sci., U.S.A.95, 4625-4629). High affinity ligand- or receptor binding oligo nucleotides habe been described by NeXstar (NX-244) and Drolet et al. (1996), Nat. Biotech 14, 1021-1025. Further, small molecules and peptides have been described.

Expression regulators have been described as anti-sense oligo nucleotides and as ribozymes-(RPI, Angiozyme™, see RPI Homepage).

Examples for delivery-/Targeting-Systems have been described as ligand/antibody-toxin-fusion-proteins or conjugates (Arora et al. (1999), Cancer Res. 59, 183-188 and Olson et al. (1997), Int. J. Cancer 73, 865-870), as endothel cell targeting of liposomes (Spragg et al. (1997), Prog. Natl. Acad. Sci, U.S.A94, 8795-8800, and as endothel cell targeting plus coagulation-induction (Ran et al., (1998), Cancer Res. 58, 4646-4653).

Small molecules which inhibit the receptor tyrosine kinase activity are for example molecules of general formula I

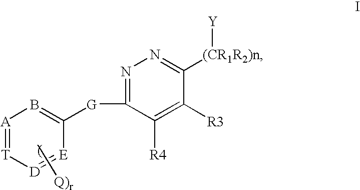

in which

r has the meaning of 0 to 2,

n has the meaning of 0 to 2;

R₃ und R₄ a) each independently from each other have the meaning of lower alkyl,

b) together form a bridge of general partial formula II,

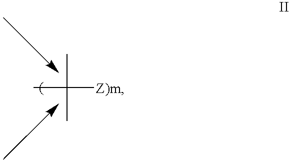

wherein the binding is via the two terminal C-atoms, and

m has the meaning of 0 to 4; or

c) together form a bridge of partial formula III

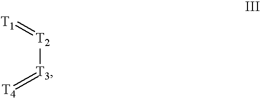

wherein one or two of the ring members T₁,T₂,T₃ ,T₄ has the meaning of nitrogen, and each others have the meaning of CH, and the bining is via the atoms T₁ and T₄;

G has the meaning of C₁-C₆-alkyl, C₂-C₆-alkylene or C₂-C₆-alkenylene; or C₂-C₆-alkylene or C₃-C₆-alkenylene, which are substituted with acyloxy or hydroxy; —CH₂—O—, —CH₂—S—, —CH₂—NH—, —CH₂—O—CH₂—, —CH₂—S—CH₂—, —CH₂—NH—CH₂, oxa (—O—), thia (—S—) or imino (—NH—),

A, B, D, E and T independently from each other have the meaning of N or CH, with the provisio that not more than three of these Substituents have the meaning of N,

Q has the meaning of lower alkyl, lower alkyloxy or halogene,

R₁ and R₂ independently from each other have the meaning of H or lower alkyl,

X has the meaning of imino, oxa or thia;

Y has the meaning of hydrogene, unsubstituted or substituted aryl, heteroaryl, or unsubstituted or substituted cycloalkyl; and

Z has the meaning of amino, mono- or disubstituted amino, halogen, alkyl, substituted alkyl, hydroxy, etherificated or esterificated hydroxy, nitro, cyano, carboxy, esterificated carboxy, alkanoyl, carbamoyl, N-mono- or N,N-disubstituted carbamoyl, amidino, guanidino, mercapto, sulfo, phenylthio, phenyl-lower-alkyl-thio, alkyl-phenyl-thio, phenylsulfinyl, phenyl-lower-alkyl-sulfinyl, alkylphenylsulfinyl, phenylsulfonyl, phenyl-lower-alkan-sulfonyl, or alkylphenylsulfonyl, whereas, if more than one rest Z is present (m≧2), the substituents Z are equal or different from each other, and wherein the bonds marked with an arrow are single or double bonds; or an N-oxide of said compound, wherein one ore more N-atoms carry an oxygene atom, or a salt thereof.

A preferred salt is the salt of an organic acid, especially a succinate.

These compounds can preferentially be used as compound I or II in the inventive pharmaceutical composition.

Compounds which stop a tyrosin phosphorylation, or the persistent angiogenese, respectively, which results in a prevention of tumor growth and tumor spread, are for example

anthranyl acid derivatives of general formula IV

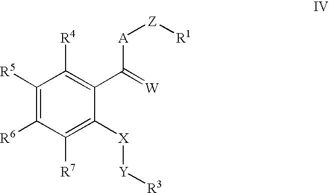

in which

A has the meaning of group ═NR²,

W has the meaning of oxygen, sulfur, two hydrogen atoms or the group ═NR⁸,

Z has the meaning of the group ═NR¹⁰ or ═N—, —N(R¹⁰)—(CH₂)_{q}-, branched or unbranched C₁₋₆-Alkyl or is the group

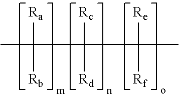

or A, Z and R¹ together form the group

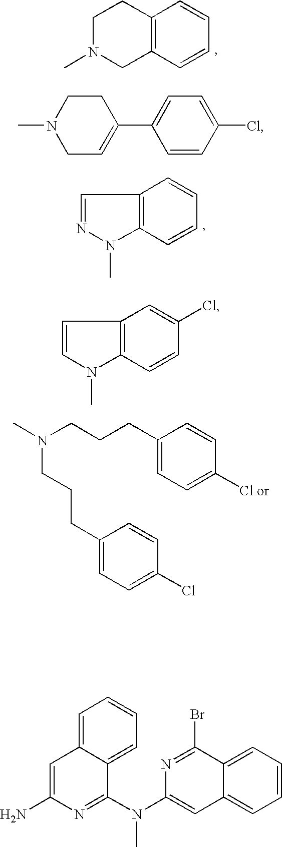

m, n and o has the meaning of 0-3,

q has the meaning of 1-6,

Rₐ, R_{b}, R_{c}, R_{d}, Rₑ, R_{f} independently from each other have the meaning of hydrogen, C₁₋₄ alkyl or the group ═NR¹⁰, and/or Rₐ and/or R_{b} together with R_{c} and or R_{d} or R_{c} together with Rₑ and/or R_{f} form a bound, or up to two of the groups Rₐ-R_{f} form a bridge with each up to 3 C-atoms with R¹ or R²,

X has the meaning of group ═NR⁹ or ═N—,

Y has the meaning of group —(CH₂)ₚ,

p has the meaning of integer 14,

R¹ has the meaning of unsubstituted or optionally substituted with one or more of halogene, C₁₋₆-alkyl, or C₁₋₆-alkyl or C₁₋₆-alkoxy, which is optionally substituted by one or more of halogen, or is unsubstituted or substituted aryl or heteroaryl,

R² has the meaning of hydrogen or C₁₋₆-alkyl, or form a bridge with up to 3 ring atoms with Rₐ-R_{f} together with Z or R₁,

R³ has the meaning of monocyclic or bicyclic aryl or heteroaryl which is unsubstituted or optionally substituted with one or more of fur halogen, C₁₋₆-alkyl, C₁₋₆-alkoxy or hydroxy,

R⁴ ,R⁵, R⁶ and R⁷ independently from each other have the meaning of hydrogen, halogen or C₁₋₆-alkoxy, C₁₋₆-alkyl or C₁₋₆-carboxyalkyl, which are unsubstituted or optionally substituted with one or more of halogen, or R⁵ and R⁶ together form the group

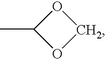

R⁸, R⁹ and R¹⁰ independently from each other have the meaning of hydrogen or C₁₋₆-alkyl, as well as their isomers and salts.

These compounds can also preferentially be used as compound I or II in the inventive pharmaceutical composition.

More preferentially compounds of genearal formula V

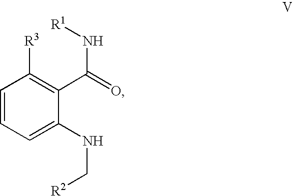

in which

R¹ has the meaning of group

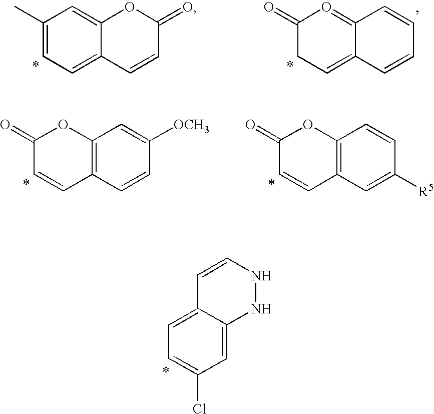

in which R⁵ is chloro, bromo or the group —OCH₃,

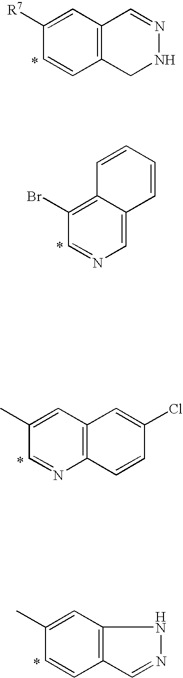

in which R⁷ is —CH₃ or chloro,

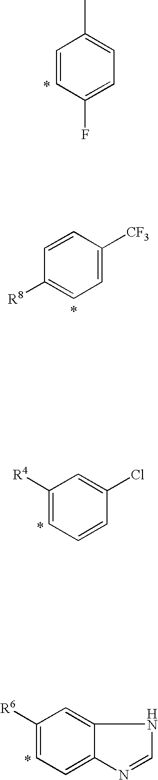

in which R⁸ is —CH₃, fluoro, in which R⁴ is fluoro, in which R⁶ is chloro or —CF₃ chloro, bromo, —CF₃, —CH₃ or chloro —N═C, —CH₃, —OCF₃ or —CH₂OH

R² has the meaning of pyridyl or the group

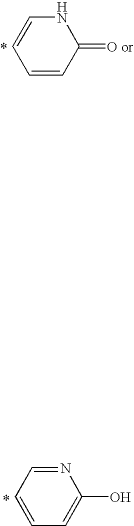

and

R³ has the meaning of hydrogen or fluoro, as well as their isomers and salts can be used as compound I or II in the inventive pharmaceutical composition.

These compounds have the same properties as already mentioned above under compound IV and can be used for the treatment of angiogeneous diseases. Compositions comprise compounds of general formulars I, IV and V, alone or in combination.

The above mentioned compounds are also claimed matter within the inventive combinations.

A further example for ligand binding inhibitors are peptides and DNA sequences coding for such peptides, which are used for the treatment of angiogeneous diseases. Such peptides and DNA sequences are disclosed in Seq. ID No. 1 to 59 of the sequence protocoll. It has been shown that Seq. ID Nos. 34 and 34a are of main interest.

Claimed matter of the instant invention are therefor pharmaceutical compositions

a) comprising one or several agents as compound I which modulate the biological function of one or several of the VEGF/VEGF receptor systems, and comprising one or several agents as compound II which modulate the biological function of one or several of the Angiopoietin/Tie receptor systems,

b) comprising one or several agents as compound I which are targeted to the endothelium via of one or several of the VEGF/VEGF receptor systems, and comprising one or several agents as compound II which modulate the biological function of one or several of the Angiopoietin/Tie receptor systems,

c) comprising one or several agents as compound I which modulates the biological function of one or several of the VEGF/VEGF receptor systems or of one or several of the Angiopoietin/Tie receptor systems and comprising one or several agents as compound II which are targeted to the endothelium,

d) comprising one or several agents as compound I which modulate the biological function of one or several of the VEGF/VEGF receptor systems, and comprising one or several agents as compound II which are targeted to the endothelium via one or several of the Angiopoietin/Tie receptor systems,

e) comprising one or several agents as compound I which are targeted to the endothelium via one or several of the VEGF/VEGF receptor systems, and comprising one or several agents as compound II which are targeted to the endothelium via one or several of the Angiopoietin/Tie receptor systems,

f) comprising one or several agents as compound I which modulate the biological function of one or several of the VEGF/VEGF receptor systems, and comprising one or several agents as compound II which are targeted to the endothelium via one or several of the VEGF/VEGF receptor systems,

g) comprising one or several agents as compound I which modulate the biological function of one or several of the Angiopoietin/Tie receptor systems, and comprising one or several agents as compound II which are targeted to the endothelium via one or several of the Angiopoietin/Tie receptor systems and

h) comprising one or several agents which interfere with both the function of one or several of the VEGF/VEGF receptor systems and the function of one or several of the Angiopoietin/Tie receptor systems.

For a sequential therapeutical application the inventive pharmaceutical compositions can be applied simultaneously or separately.

The inventive compositions comprise as compound I or as compound II at least one of

a) compounds which inhibit receptor tyrosine kinase activity,

b) compounds which inhibit ligand binding to receptors,

c) compounds which inhibit activation of intracellular signal pathways of the receptors,

d) compounds which inhibit or activate expression of a ligand or of a receptor of the VEGF or Tie receptor system,

e) delivery systems, such as antibodies, ligands, high-affinity binding oligonucleotides or oligopeptides, or liposomes, which target cytotoxic agents or coagulation-inducing agents to the endothelium via recognition of VEGF/VEGF receptor or Angiopoietin/Tie receptor systems,

f) delivery systems, such as antibodies, ligands, high-affinity binding oligonucleotides or oligopeptides, or liposomes, which are targeted to the endothelium and induce necrosis or apoptosis.

These compositions are also claimed matter of the present invention.

Also claimed matter of the present invention are pharmaceutical compositions which comprise as compound I and/or II at least one of Seq. ID Nos. 1-59. Of most value are pharmaceutical compositions, which comprise as compound I and/or II Seq. ID Nos. 34a und pharmaceutical compositions according to claims which comprise as compound I and/or II at least one of sTie2, mAB 4301-42-35, scFv-tTF and/or L19 scFv-tTF conjugate.

Further preferred matter of the present invention are pharmaceutical compositions, which comprise as compound I and/or II at least one small molecule of general formula I, general formula IV and/or general formula V.

The most preferred compound which can be used as compound I or II in the inventive composition is (4-Chlorophenyl)[4-(4-pyridylmethyl)-phthalazin-1-yl]ammonium hydrogen succinate.

Therefore, claimed matter of the present invention are also pharmaceutical compositions, which comprise as compound I (4-Chlorophenyl)[4-(4-pyridylmethyl)-phthalazin-1-yl]ammonium hydrogen succinate, sTie2, mAB 4301-42-35, scFv-tTF and/or L19 scFv-tTF conjugate, and as compound II (4-Chlorophenyl)[4-(4-pyridylmethyl)-phthalazin-1-yl]ammonium hydrogen succinatesTie2, mAB 4301-42-35, scFv-tTF and/or L19 scFv-tTF conjugate, with the provisio that compound I is not identically to compound II, and most preferred pharmaceutical compositions, which comprise as compound I (4-Chlorophenyl)[4-(4-pyridylmethyl)-phthalazin-1-yl]ammonium hydrogen succinate and as compound II sTie2, mAB 4301-42-35, scFv-tTF and/or L19 scFv-tTF conjugate; pharmaceutical compositions, which comprise as compound I mAB 4301-42-35 and as compound II sTie2, and/or scFv-tTF conjugate; pharmaceutical compositions, which comprise as compound I scFv-tTF conjugate and as compound II sTie2 and/or mAB 4301-42-35; pharmaceutical compositions, which comprise as compound I L19 scFv-tTF conjugate and as compound II sTie2.

The small molecule compounds, proteins and DNA's expressing proteins, as mentioned above can be used as medicament alone, or in form of formulations for the treatment of tumors, cancers, psoriasis, arthritis, such as rheumatoide arthritis, hemangioma, angiofribroma, eye diseases, such as diabetic retinopathy, neovascular glaukoma, kidney diseases, such as glomerulonephritis, diabetic nephropathy, maligneous nephrosclerose, thrombic microangiopatic syndrome, transplantation rejections and glomerulopathy, fibrotic diseases, such as cirrhotic liver, mesangial cell proliferative diseases, artheriosclerosis and damage of nerve tissues.

The treatment of the damaged nerve tissues with the inventive combination hinders the rapid formation of scars at the damaged position. Thus, there is no scar formation before the axons communicate with each other. Therefore a reconstruction of the nerve bindings is much more easier.

Further, the inventive combinations can be used for suppression of the ascites formation in patients. It is also possible to suppress VEGF oedemas.

For the use of the inventive combinations as medicament the compounds will be formulated as pharmaceutical composition. Said formulation comprises beside the active compound or compounds acceptable pharmaceutically, organically or inorganically inert carriers, such as water, gelatine, gum arabic, lactose, starch, magnesium stearate, talcum, plant oils, polyalkylene glycols, etc. Said pharmaceutical preparations can be applied in solid form, such as tablets, pills, suppositories, capsules, or can be applied in fluid form, such as solutions, suspensions or emulsions.

If necessary, the compositions additionally contain additives, such as preservatives, stabilizer, detergents or emulgators, salts for alteration of the osmotic pressure and/or buffer.

These uses are also claimed matter of the instant invention, as well as the formulations of the active compounds

For parenteral application especially injectable solutions or suspensions are suitable, especially hydrous solutions of the active compound in polyhydroxyethoxylated castor-oil are suitable.

As carrier also additives can be used, such as salts of the gallic acid or animal or plant phospholipids, as well as mixtures thereof, and liposomes or ingredients thereof.

For oral application especially suitable are tablets, pills or capsules with talcum and/or hydrocarbon carriers or binders, such as lactose, maize or potato starch. The oral application can also be in form of a liquid, such as juice, which optionally contains a sweetener.

The dosis of the active compound differs depending on the application of the compound, age and weight of the patient, as well as the form and the progress of the disease.

The daily dosage of the active compound is 0.5-1000 mg, especially 50-200 mg. The dosis can be applied as single dose or as two or more daily dosis.

These formulations and application forms are also part of the instant invention.

Combined functional interference with VEGF/VEGF receptor systems and with Angiopoietin/Tie receptor systems can be performed simultaneously, or in sequential order such that the biological response to interference with one ligand/receptor system overlaps with the biological response to interference with a second ligand/receptor system. Alternatively, combined functional interference with VEGF/VEGF receptor systems or with Angiopoietin/Tie receptor systems and targeting of cytotoxic agents via VEGF/VEGF receptor systems or via Angiopoietin/Tie receptor systems can be performed simultaneously, or in sequential order such that the biological response to functional interference with a ligand/receptor system overlaps in time with targeting of cytotoxic agents.

The invention is also directed to a substance which functional interferes with both VEGF/VEGF receptor systems and Angiopoietin/Tie receptor systems, or which are targeted via both VEGF/VEGF receptor systems and Angiopoietin/Tie receptor systems.

VEGF/VEGF receptor systems include the ligands VEGF-A, VEGF-B, VEGF-C, VEGF-D, PIGF, and the receptor tyrosine kinases VEGF-R1 (Flt1), VEGF-R2 (KDR/Flk1), VEGF-R3 (Flt4), and their co-receptors (i.e. neuropilin-1). Angiopoietin/Tie receptor systems include Ang1, Ang2, Ang3/Ang4, and angiopoietin related polypeptides which bind to Tie1 or to Tie2, and the receptor tyrosine kinases Tie1 and Tie2.

Phamaceutical compositions of the present invention can be used for medicinal purposes. Such diseases are, for example, cancer, cancer metastasis, angiogenesis including retinopathy and psoriasis. Pharmaceutical compositions of the present invention can be applied orally, parenterally, or via gene therapeutic methods.

Therefor the present invention also concerns the use of pharmaceutical compositions for the production of a medicament for the treatment of tumors, cancers, psoriasis, arthritis, such as rheumatoide arthritis, hemangioma, angiofribroma, eye diseases, such as diabetic retinopathy, neovascular glaukoma, kidney diseases, such as glomerulonephritis, diabetic nephropathie, maligneous nephrosclerosis, thrombic microangiopatic syndrome, transplantation rejections and glomerulopathy, fibrotic diseases, such as cirrhotic liver, mesangial cell proliferative diseases, artheriosclerosis, damage of nerve tissues, suppression of the ascites formation in patients and suppression of VEGF oedemas.

The following examples demonstrate the feasability of the disclosed invention, without restricting the inventon to the disclosed examples.

### EXAMPLE 1

Superior effect on inhibition of tumor growth via combination of inhibition of the VEGF A/VEGF receptor system together with functional interference with the Angiopoietin/Tie2 receptor system over separate modes of intervention was demonstrated in an A375v human melanoma xenograft model.

Human melanoma cell line A375v was stably transfected to overexpress the extracellular ligand-neutralizing domain of human Tie2 receptor tyrosine kinase (sTie2; compound II) (Siemeister et al., Cancer Res. 59, 3185-3191, 1999). For control, A375v cells were stably transfected with the empty expression vector (A375v/pCEP). Swiss nu/nu mice were s.c. injected with 1×10⁶ transfected A375v/sTie2 or A375v/pCEP tumor cells, respectively. Animals receiving compound I were treated for up to 38 days with daily oral doses of 50 mg/kg of the VEGF receptor tyrosine kinase inhibitor (4-Chlorophenyl)[4-(4-pyridylmethyl)-phthalazin-1-yl]ammonium hydrogen succinate (Wood et al., Cancer Res. 60, 2178-2189, 2000). Various modes of treatment are described in Table 1. Tumor growth was determined by caliper measurement of the largest diameter and its perpendicular.

TABLE 1

| | mode of treatment | |
|---|---|---|
| | (4-Chlorophenyl)[4-(4-pyridylmethyl)- | |
| | phthalazin-1-yl]ammonium hydrogen | sTie2 |
| treatment group | succinate (compound I) | (compound II) |
| Group 1: | − | − |
| A375v/pCEP | | |
| Group 2: | + | − |
| A375v/pCEP | | |
| Group 3: | − | + |
| A375v/sTie2 | | |
| Group 4: | + | + |
| A375v/sTie2 | | |

Tumors derived from A375v/pCEP control cells reached a size of approx. 250 mm² (mean area) within 24 days (FIG. 1) without treatment (group 1). Separate treatment with the VEGF receptor inhibitor (4-Chlorophenyl)[4-(4-pyridylmethyl)-phthalazin-1-yl]ammonium hydrogen succinate (compound I, treatment group 2) or separate interference with Angiopoietin/Tie2 receptor system by means of expression of sTie2 (compound II, treatment group 3) delayed growth of tumors to a size of approx. 250 mm² to 31 days, respectively. Combination of interference with the Angiopoietin/Tie2 system by means of expression of sTie2 and of interference with the VEGF/VEGF receptor system by means of the kinase inhibitor (4-Chlorophenyl)[4-(4-pyridylmethyl)-phthalazin-1-yl]ammonium hydrogen succinate (compound I+compound II, treatment group 4) delayed growth of the tumors to a size of approx. 250 mm² to 38 days.

This result clearly demonstrates the superior effect of a combination of interference with the VEGF-A/VEGF receptor system and the Angiopoietin/Tie2 receptor system over separate modes of intervention.

### EXAMPLE 2

Combination of functional interference with the Angiopoietin/Tie2 receptor system and neutralization of VEGF-A is superior to separate modes of intervention in inhibition of tumor growth.

Tumors derived from A375v/sTie2 cells and from A375v/pCEP cells were induced in nude mice as described in example 1. Animals receiving compound I were treated twice weekly over a period of time of 4 weeks with intraperitoneal doses of 200 μg of the VEGF-A-neutralizing monoclonal antibody (mAb) 4301-42-35 (Schlaeppi et al., J. Cancer Res. Clin. Oncol. 125, 336-342, 1999). Various modes of treatment are descibed in Table 2. Animals were sacrificed for ethical reasons when tumors of group 1 exceeded a volume of approx. 1000 mm³. Tumor growth was determined by caliper measurement of the largest diameter and its perpendicular.

TABLE 2

mode of treatment

| | mAb 4301-42-35 | sTie2 |
|---|---|---|
| treatment group | (compound I) | (compound II) |
| Group 1: | − | − |
| A375v/pCEP | | |
| Group 2: | + | − |
| A375v/pCEP | | |
| Group 3: | − | + |
| A375v/sTie2 | | |
| Group 4: | + | + |
| A375v/sTie2 | | |

Tumors derived from A375v/pCEP control cells reached a size of approx. 1000 mm³ within 28 days (FIG. 2) without treatment (group 1). Tumors treated with the VEGF-A-neutralizing mAb 4301-42-35 (compound I, treatment group 2) grew to a volume of approx. 450 mm³ within 28 days. Interference with Angiopoietin/Tie2 receptor system by means of expression of sTie2 (compound II, treatment group 3) reduced growth of tumors within 28 day to a volume of approx. 600 mm², respectively. Combination of interference with the Angiopoietin/Tie2 system by means of expression of sTie2 and neutralizing of VEGF-A by means of the mAb 4301-42-35 (compound I+compound II, treatment group 4) resulted in a inhibition of tumor growth to a volume of approx. 250 mm³ within 28 days.

The superior effect of a combination of neutralization of VEGF-A and functional interference with the Angiopoietin/Tie2 receptor system over separate modes of intervention is clearly shown.

### EXAMPLE 3

Combination of functional interference with the Angiopoietin/Tie2 receptor system and targeting of a coagulation-inducing protein via the VEGF/VEGF receptor system is superior to separate modes of intervention in inhibition of tumor growth.

Tumors derived from A375v/sTie2 cells and from A375v/pCEP cells were induced in nude mice as described in example 1. A single chain antibody (scFv) specifically recognizing the human VEGF-A/VEGF receptor I complex (WO 99/19361) was expressed in E. coli and conjugated to coagulation-inducing recombinant human truncated tissue factor (tTF) by methods descibed by Ran et al. (Cancer Res. 58, 4646-4653, 1998). When tumors reached a size of approx. 200 mm³ animals receiving compound I were treated on day 0 and on day 4 with intravenous doses of 20 μg of the scFv-tTF conjugate. Various modes of treatment are described in Table 3. Animals were sacrificed for ethical reasons when tumors of group 1 exceeded a volume of approx. 1000 mm³. Tumor growth was determined by caliper measurement of the largest diameter and its perpendicular.

TABLE 3

mode of treatment

| | scFv-tTF conjugate | sTie2 |
|---|---|---|
| treatment group | (compound I) | (compound II) |
| Group 1: | − | − |
| A375v/pCEP | | |
| Group 2: | + | − |
| A375v/pCEP | | |
| Group 3: | − | + |
| A375v/sTie2 | | |
| Group 4: | + | + |
| A375v/sTie2 | | |

Tumors derived from A375v/pCEP control cells reached a size of approx. 1000 mm³ within 28 days (FIG. 3) without treatment (group 1). Tumors treated with the coagulation-inducting tTF targeted to the VEGF-A/VEGF receptor I complex via the scFv-tTF conjugate (compound I, treatment group 2) grew to a volume of approx. 500 mm³ within 28 days. Interference with Angiopoietin/Tie2 receptor system by means of expression of sTie2 (compound II, treatment group 3) reduced growth of tumors within 28 day to a volume of approx. 600 mm², respectively. Combination of interference with the Angiopoietin/Tie2 system by means of expression of sTie2 and of targeting the VEGF receptor complex (compound I+compound II, treatment group 4) resulted in a inhibition of tumor growth to a volume of approx. 300 mm³ within 28 days.

The superior effect of a combination of targeting of the coagulation-inducting tTF to the VEGF-A/VEGF receptor I complex and functional interference with the Angiopoietin/Tie2 receptor system over separate modes of intervention is clearly shown. Similar effects can be expected upon targeting of cytotoxic agents to VEGF/VEGF receptor systems.

### EXAMPLE 4

Combination of functional interference with the VEGF/VEGF receptor system and targeting of a coagulation-inducing protein via the VEGF/VEGF receptor system is superior to separate modes of intervention in inhibition of tumor growth.

Tumors derived from A375v/pCEP cells were induced in nude mice as described in example 1. Animals receiving compound I were treated for up to 28 days with daily oral doses of 50 mg/kg of the VEGF receptor tyrosine kinase inhibitor (4-Chlorophenyl)[4-(4-pyridylmethyl)-phthalazin-1-yl]ammonium hydrogen succinate (Wood et al., Cancer Res. 60, 2178-2189, 2000). Compound II consists of a single chain antibody (scFv) specifically recognizing the human VEGF-A/VEGF receptor I complex (WO 99/19361) which was expressed in E. coli and conjugated to coagulation-inducing recombinant human truncated tissue factor (tTF) by methods descibed by Ran et al. (Cancer Res. 58, 4646-4653, 1998). When tumors reached a size of approx. 200 mm³ animals receiving compound II were treated on day 0 and on day 4 with intravenous doses of 20 μg of the scFv-tTF conjugate. Various modes of treatment are described in Table 4. Animals were sacrificed for ethical reasons when tumors of group 1 exceeded a volume of approx. 1000 mm³. Tumor growth was determined by caliper measurement of the largest diameter and its perpendicular.

TABLE 4

| | mode of treatment | |
|---|---|---|
| | (4-Chlorophenyl)[4-(4-pyridylmethyl)- | |
| | phthal-azin-1-yl]ammonium hydrogen | scFv-tTF |
| | succinate | conjugate |
| treatment group | (compound I) | (compound II) |
| Group 1: | − | − |
| A375v/pCEP | | |
| Group 2: | + | − |
| A375v/pCEP | | |
| Group 3: | − | + |
| A375v/pCEP | | |
| Group 4: | + | + |
| A375v/pCEP | | |

Tumors derived from A375v/pCEP control cells reached a size of approx. 1000 mm³ within 28 days (FIG. 4) without treatment (group 1). Separate treatment with the VEGF receptor inhibitor (4-Chlorophenyl)[4-(4-pyridylmethyl)-phthalazin-1-yl]ammonium hydrogen succinate (compound I, treatment group 2) resulted in a reduction of the tumor volumes to approx. 550 mm³. Tumors treated with the coagulation-inducting tTF targeted to the VEGF-A/VEGF receptor I complex via the scFv-tTF conjugate (compound II, treatment group 3) grew to a volume of approx. 500 mm³ within 28 days. Combination of inhibition of VEGF receptor tyrosine kinase by means of (4-Chlorophenyl)[4-(4-pyridylmethyl)-phthalazin-1-yl]ammonium hydrogen succinate and of targeting the VEGF receptor complex (compound I+compound II, treatment group 4) resulted in a inhibition of tumor growth to a volume of approx. 400 mm³ within 28 days.

The superior effect of a combination of targeting of the coagulation-inducting tTF to the VEGF-A/VEGF receptor I complex and functional interference with the VEGF/VEGF receptor system over separate modes of intervention is clearly shown. Similar effects can be expected upon targeting of cytotoxic agents to Angiopoietin/Tie receptor systems.

### EXAMPLE 5

Combination of functional interference with the Angiopoietin/Tie2 receptor system and endothelium-specific targeting of a coagulation-inducing protein is superior to separate modes of intervention in inhibition of tumor growth.

Tumors derived from A375v/sTie2 cells and from A375v/pCEP cells were induced in nude mice as described in example 1. A fusion protein (L19 scFv-tTF) consisting of L19 single chain antibody specifically recognizing the oncofoetal ED-B domain of fibronectin and the extracellular domain of tissue factor was expressed in E. coli as described by Nilsson et al. (Nat. Med., in press). Further, L19 scFv-tTF data have been represented by D. Neri and F. Nilsson (Meeting “Advances in the application of monoclonal antibodies in clinical oncology”, Samos, Greece, 31. May-2. June 2000). When tumors reached a size of approx. 200 mm³ animals receiving compound I were treated with a single intravenous dose of 20 μg of L19 scFv-tTF in 200 μl saline. Various modes of treatment are described in Table 5. Animals were sacrificed for ethical reasons when tumors of group 1 exceeded a volume of approx. 1000 mm³. Tumor growth was determined by caliper measurement of the largest diameter and its perpendicular.

TABLE 5

mode of treatment

| | L19 scFv-tTF | sTie2 |
|---|---|---|
| treatment group | (compound I) | (compound II) |
| Group 1: | − | − |
| A375v/pCEP | | |
| Group 2: | + | − |
| A375v/pCEP | | |
| Group 3: | − | + |
| A375v/sTie2 | | |
| Group 4: | + | + |
| A375v/sTie2 | | |

Tumors derived from A375v/pCEP control cells reached a size of approx. 1000 mm³ within 28 days (FIG. 5) without treatment (group 1). Tumors treated with the coagulation-inducting L19 scFv-tTF (compound I, treatment group 2) grew to a volume of approx. 450 mm³ within 28 days. Interference with Angiopoietin/Tie2 receptor system by means of expression of sTie2 (compound II, treatment group 3) reduced growth of tumors within 28 day to a volume of approx. 600 mm², respectively. Combination of interference with the Angiopoietin/Tie2 system by means of expression of sTie2 and of targeting the endothelium with L19 scFv-tTF (compound I+compound II, treatment group 4) resulted in a inhibition of tumor growth to a volume of approx. 250 mm³ within 28 days.

The superior effect of a combination of targeting of L19 scFv-tTF to the endothelium and functional interference with the Angiopoietin/Tie2 receptor system over separate modes of intervention is clearly shown.

### EXAMPLE 6

Combination of functional interference with the VEGF/VEGF receptor system and endothelium-specific targeting of a coagulation-inducing protein is superior to separate modes of intervention in inhibition of tumor growth.

Tumors derived from A375v/pCEP cells were induced in nude mice as described in example 1. Animals receiving compound I were treated for up to 28 days with daily oral doses of 50 mg/kg of the VEGF receptor tyrosine kinase inhibitor (4-Chlorophenyl)[4-(4-pyridylmethyl)-phthalazin-1-yl]ammonium hydrogen succinate (Wood et al., Cancer Res. 60, 2178-2189, 2000). Compound II consists of L19 scFv-tTF fusion protein as described in example 5. When tumors reached a size of approx. 200 mm³ animals receiving compound II were treated with a single intravenous dose of 20 μg of L19 scFv-tTF in 200 μl saline. Various modes of treatment are described in Table 6. Animals were sacrificed for ethical reasons when tumors of group 1 exceeded a volume of approx. 1000 mm³. Tumor growth was determined by caliper measurement of the largest diameter and its perpendicular.

TABLE 6

| | mode of treatment | |
|---|---|---|
| | (4-Chlorophenyl)[4-(4-pyridylmethyl)- | |
| | phthal-azin-1-yl]ammonium hydrogen | |
| | succinate | L19 scFv-tTF |
| treatment group | (compound I) | (compound II) |
| Group 1: | − | − |
| A375v/pCEP | | |
| Group 2: | + | − |
| A375v/pCEP | | |
| Group 3: | − | + |
| A375v/pCEP | | |
| Group 4: | + | + |
| A375v/pCEP | | |

Tumors derived from A375v/pCEP control cells reached a size of approx. 1000 mm³ within 28 days (FIG. 6) without treatment (group 1). Separate treatment with the VEGF receptor inhibitor (4-Chlorophenyl)[4-(4-pyridylmethyl)-phthalazin-1-yl]ammonium hydrogen succinate (compound I, treatment group 2) resulted in a reduction of the tumor volumes to approx. 550 mm³. Tumors treated with the coagulation-inducting L19 scFv-tTF targeted to the endothelium (compound II, treatment group 3) grew to a volume of approx. 450 mm³ within 28 days. Combination of inhibition of VEGF receptor tyrosine kinase by means of (4-Chlorophenyl)[4-(4-pyridylmethyl)-phthalazin-1-yl]ammonium hydrogen succinate and of targeting the VEGF receptor complex (compound I+compound II, treatment group 4) resulted in a inhibition of tumor growth to a volume of approx. 200 mm³ within 28 days.

The superior effect of a combination of targeting of L19 scFv-tTF to the endothelium and functional interference with the VEGF/VEGF receptor system over separate modes of intervention is clearly shown.

### DESCRIPTION OF THE FIGURES

FIG. 1 shows the superior effect of combination of interference with VEGF/VEGF receptor system by means of an specific tyrosine kinase inhibitor and with the Angiopoietin/Tie2 receptor system by means of a soluble receptor domain on inhibition of tumor growth (treatment modes of groups 1-4 are given in Table 1). The abbreviations have the following meaning:

| | |
|---|---|
| mock, con. = | treatment group 1 |
| mock + VEGF-A = | treatment group 2 |
| sTIE2-cl13 = | treatment group 3 |
| sTIE2-cl13 + VEGF-A = | treatment group 4 |

FIG. 2 shows the superior effect on tumor growth inhibition of combination of VEGF-neutralization and functional interference with Angiopoietin/Tie2 receptor system over separate modes of intervention (treatment modes of groups 1-4 are given in Table 2).

FIG. 3 shows the superior effect on tumor growth inhibition of combination of targeting of the coagulation-inducing tTF to the VEGF/VEGF receptor I complex via a scFv-tTF conjugate and functional interference with Angiopoietin/Tie2 receptor system over separate modes of intervention (treatment modes of groups 1-4 are given in Table 3).

FIG. 4 shows the superior effect on tumor growth inhibition of combination of targeting of the coagulation-inducing tTF to the VEGF/VEGF receptor I complex via a scFv-tTF conjugate and functional interference with VEGF/VEGF receptor system by means of the VEGF receptor tyrosine kinase inhibitor (4-Chlorophenyl)[4-(4-pyridylmethyl)-phthalazin-1-yl]ammonium hydrogen succinate over separate modes of intervention (treatment modes of groups 1-4 are given in Table 4).

FIG. 5 shows the superior effect on tumor growth inhibition of combination of targeting of the coagulation-inducing L19 scFv-tTF fusion protein to the endothelium and functional interference with Angiopoietin/Tie2 receptor system over separate modes of intervention (treatment modes of groups 1-4 are given in Table 5).

FIG. 6 shows the superior effect on tumor growth inhibition of combination of targeting of the coagulation-inducing L19 scFv-tTF fusion protein to the endothelium and functional interference with VEGF/VEGF receptor system by means of the VEGF receptor tyrosine kinase inhibitor (4-Chlorophenyl)[4-(4-pyridylmethyl)-phthalazin-1-yl]ammonium hydrogen succinate over separate modes of intervention (treatment modes of groups 1-4 are given in Table 6).

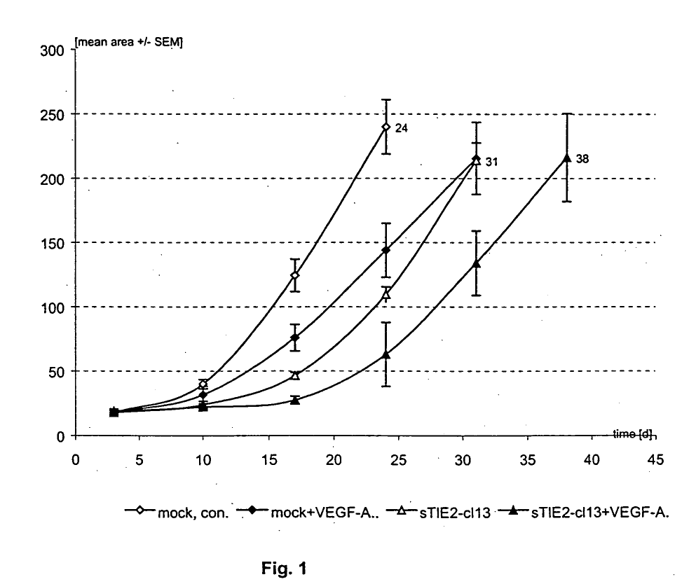

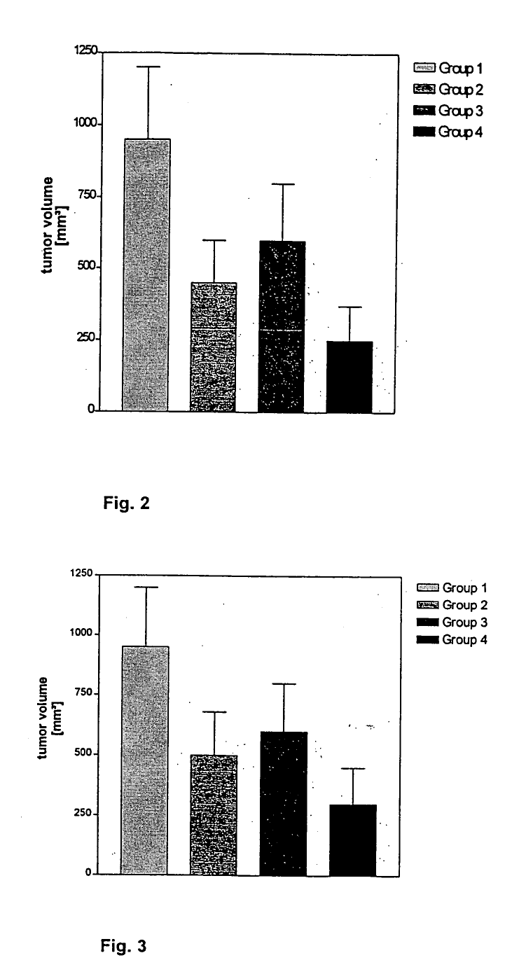

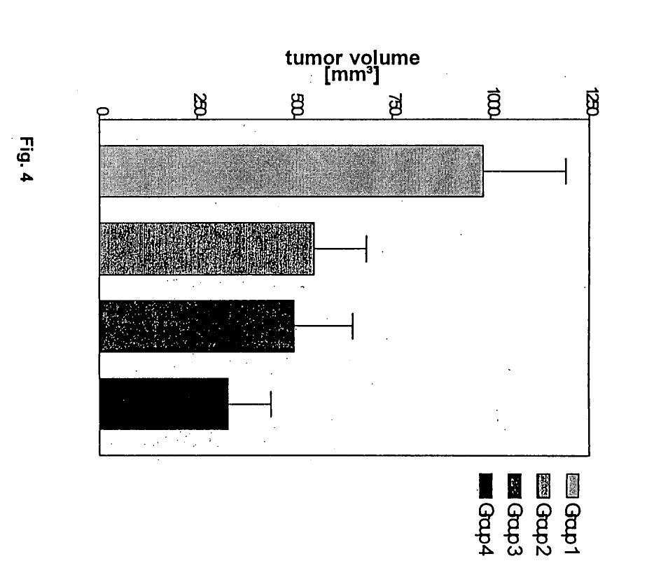

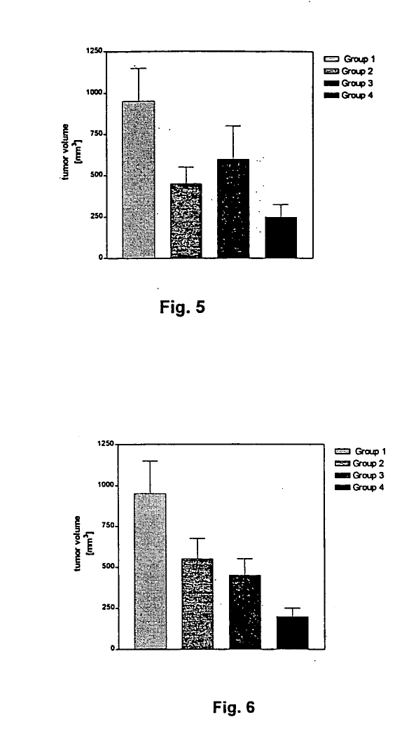

1. Pharmaceutical compositions comprising one or several agents as compound I which modulate the biological function of one or several of the VEGF/VEGF receptor systems, and comprising one or several agents as compound II which modulate the biological function of one or several of the Angiopoietin/Tie receptor systems.

2. Pharmaceutical compositions comprising one or several agents as compound I which are targeted to the endothelium via of one or several of the VEGF/VEGF receptor systems, and comprising one or several agents as compound II which modulate the biological function of one or several of the Angiopoietin/Tie receptor systems.

3. Pharmaceutical compositions comprising one or several agents as compound I which modulates the biological function of one or several of the VEGF/VEGF receptor systems or of one or several of the Angiopoietin/Tie receptor systems and comprising one or several agents as compound II which are targeted to the endothelium.

4. Pharmaceutical compositions comprising one or several agents as compound I which modulate the biological function of one or several of the VEGF/VEGF receptor systems, and comprising one or several agents as compound II which are targeted to the endothelium via one or several of the Angiopoietin/Tie receptor systems.

5. Pharmaceutical compositions comprising one or several agents as compound I which are targeted to the endothelium via one or several of the VEGF/VEGF receptor systems, and comprising one or several agents as compound II which are targeted to the endothelium via one or several of the Angiopoietin/Tie receptor systems.

6. Pharmaceutical compositions comprising one or several agents as compound I which modulate the biological function of one or several of the VEGF/VEGF receptor systems, and comprising one or several agents as compound II which are targeted to the endothelium via one or several of the VEGF/VEGF receptor systems.

7. Pharmaceutical compositions comprising one or several agents as compound I which modulate the biological function of one or several of the Angiopoietin/Tie receptor systems, and comprising one or several agents as compound II which are targeted to the endothelium via one or several of the Angiopoietin/Tie receptor systems.

8. Pharmaceutical compositions comprising one or several agents which interfere with both the function of one or several of the VEGF/VEGF receptor systems and the function of one or several of the Angiopoietin/Tie receptor systems.

9. Pharmaceutical compositions according to claims 1-8 which are intended for simultaneous or separate sequential therapeutical application.

10. Pharmaceutical compositions according to claims 1-8 which comprise as compound I at least one of
a) compounds which inhibit receptor tyrosine kinase activity,
b) compounds which inhibit ligand binding to receptors,
c) compounds which inhibit activation of intracellular signal pathways of the receptors,
d) compounds which inhibit or activate expression of a ligand or of a receptor of the VEGF or Tie receptor system,
e) delivery systems, such as antibodies, ligands, high-affinity binding oligonucleotides or oligopeptides, or liposomes, which target cytotoxic agents or coagulation-inducing agents to the endothelium via recognition of VEGF/VEGF receptor or Angiopoietin/Tie receptor systems,
f) delivery systems, such as antibodies, ligands, high-affinity binding oligonucleotides or oligopeptides, or liposomes, which are targeted to the endothelium and induce necrosis or apoptosis.

11. Pharmaceutical compositions according to claims 1-8 which comprise as compound II at least one of
g) compounds which inhibit receptor tyrosine kinase activity,
h) compounds which inhibit ligand binding to receptors,
i) compounds which inhibit activation of intracellular signal pathways of the receptors,
j) compounds which inhibit or activate expression of a ligand or of a receptor of the VEGF or Tie receptor system,
k) delivery systems, such as antibodies, ligands, high-affinity binding oligonucleotides or oligopeptides, or liposomes, which target cytotoxic agents or coagulation-inducing agents to the endothelium via recognition of VEGF/VEGF receptor or Angiopoietin/Tie receptor systems,
l) delivery systems, such as antibodies, ligands, high-affinity binding oligonucleotides or oligopeptides, or liposomes, which are targeted to the endothelium and induce necrosis or apoptosis.

12. Pharmaceutical compositions according to claims 1-11 which comprise as compound I and/or II at least one of Seq. ID Nos. 1-59.

13. Pharmaceutical compositions according to claims 1-11 which comprise as compound I and/or II Seq. ID Nos. 34a

14. Pharmaceutical compositions according to claims 1-11 which comprise as compound I and/or II at least one of sTie2, mAB 4301-42-35, scFv-tTF and/or L19 scFv-tTF conjugate.

15. Pharmaceutical compositions according to claims 1-11 which comprise as compound I and/or II at least one small molecule of genaral formula I 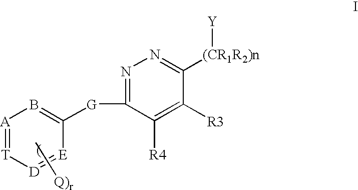
in which
r has the meaning of 0 to 2,
n has the meaning of 0 to 2;
R₃ und R₄ a) each independently from eaxh other have the meaning of lower alkyl,
b) together form a bridge of general partial formula II, 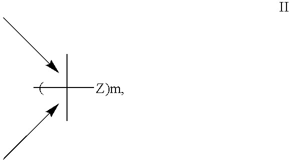
wherein the binding is via the two terminal C-atoms, and
m has the meaning of 0 to 4; or
c) together form a bridge of partial formula III 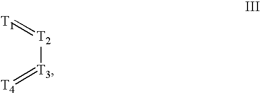
wherein one or two of the ring members T₁,T₂,T₃,T₄ has the meaning of nitrogen, and each others have the meaning of CH, and the bining is via the atoms T₁ and T₄;
G has the meaning of C₁-C₆-alkyl, C₂-C₆-alkylene or C₂-C₆-alkenylene; or C₂-C₆-alkylene or C₃-C₆-alkenylene, which are substituted with acyloxy or hydroxy; —CH₂—O—, —CH₂—S—, —CH₂—NH—, —CH₂—O—CH₂—, —CH₂—S—CH₂—, —CH₂—NH—CH₂, oxa (—O—), thia (—S—) or imino (—NH—),
A, B, D, E and T independently from each other have the meaning of N or CH, with the provisio that not more than three of these Substituents have the meaning of N,
Q has the meaning of lower alkyl, lower alkyloxy or halogene,
R¹ and R₂ independently from each other have the meaning of H or lower alkyl,
X has the meaning of imino, oxa or thia;
Y has the meaning of hydrogene, unsubstituted or substituted aryl, heteroaryl, or unsubstituted or substituted cycloalkyl; and
Z has the meaning of amino, mono- or disubstituted amino, halogen, alkyl, substituted alkyl, hydroxy, etherificated or esterificated hydroxy, nitro, cyano, carboxy, esterificated carboxy, alkanoyl, carbamoyl, N-mono- or N,N-disubstituted carbamoyl, amidino, guanidino, mercapto, sulfo, phenylthio, phenyl-lower-alkyl-thio, alkyl-phenyl-thio, phenylsulfinyl, phenyl-lower-alkyl-sulfinyl, alkylphenylsulfinyl, phenylsulfonyl, phenyl-lower-alkan-sulfonyl, or alkylphenylsulfonyl, whereas, if more than one rest Z is present (m≧2), the substituents Z are equal or different from each other, and wherein the bonds marked with an arrow are single or double bonds; or an N-oxide of said compound, wherein one ore more N-atoms carry an oxygene atom, or a salt thereof,
and/or a compound of genaral formula IV 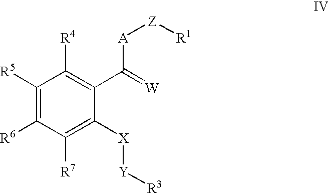
in which
A has the meaning of group ═NR²,
W has the meaning of oxygen, sulfur, two hydrogen atoms or the group ═NR⁸,
Z has the meaning of the group ═NR¹⁰ or ═N—, —N(R¹⁰)—(CH₂)_{q}-, branched or unbranched C₁₋₆-Alkyl or is the group 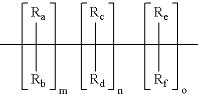
or A, Z and R¹ together form the group 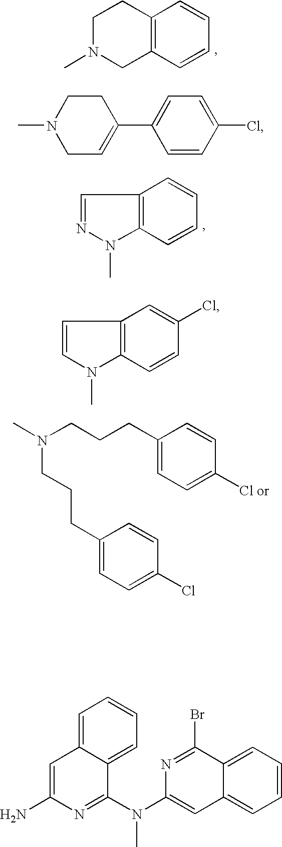
m, n and o has the meaning of 0-3,
q has the meaning of 1-6, Rₐ, R_{b}, R_{c}, R_{d}, Rₑ, R_{f} independently from each other have the meaning of hydrogen, C₁₋₄ alkyl or the group ═NR¹⁰, and/or Rₐ and/or R_{b} together with R_{c} and or R_{d} or R_{c} together with Rₑ and/or R_{f} form a bound, or up to two of the groups Rₐ-R_{f} form a bridge with each up to 3 C-atoms with R¹ or R²,
X has the meaning of group ═NR⁹ or ═N—,
Y has the meaning of group —(CH₂)ₚ,
p has the meaning of integer 1-4,
R¹ has the meaning of unsubstituted or optionally substituted with one or more of halogene, C₁₋₆-alkyl, or C₁₋₆-alkyl or C₁₋₆alkoxy, which is optionally substituted by one or more of halogen, or is unsubstituted or substituted aryl or heteroaryl,
R² has the meaning of hydrogen or C₁₋₆-alkyl, or form a bridge with up to 3 ring atoms with Rₐ-R_{f} together with Z or R₁,
R³ has the meaning of monocyclic or bicyclic aryl or heteroaryl which is unsubstituted or optionally substituted with one or more of für halogen, C₁₋₆-alkyl, C₁₋₆-alkoxy or hydroxy,
R⁴ ,R⁵, R⁶ and R⁷ independently from each other have the meaning of hydrogen, halogene or C₁₋₆-alkoxy, C₁₋₆-alkyl or C₁₋₆-carboxyalkyl, which are unsubstituted or optionally substituted with one or more of halogene, or R⁵ and R⁶ together form the group 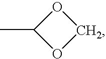
R⁸, R⁹ and R¹⁰ independently from each other have the meaning of hydrogen or C₁₋₆-alkyl,
as well as their isomers and salts,
and/or a compound of general formula V 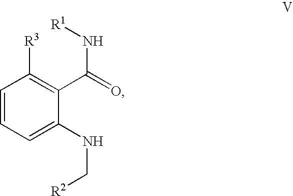
in which
R¹ has the meaning of group 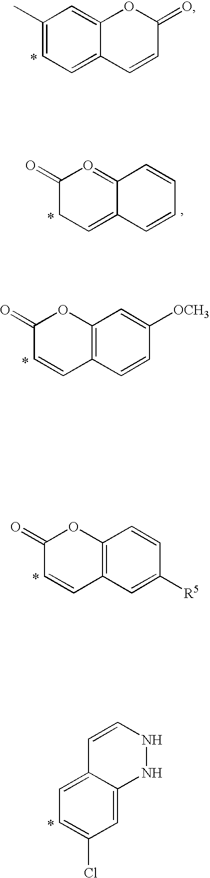
in which R⁵ is chloro, bromo or the group —OCH₃, 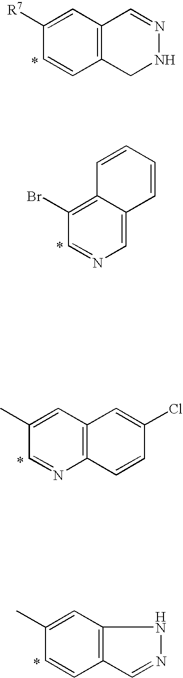
in which R⁷ is —CH₃ or chloro, 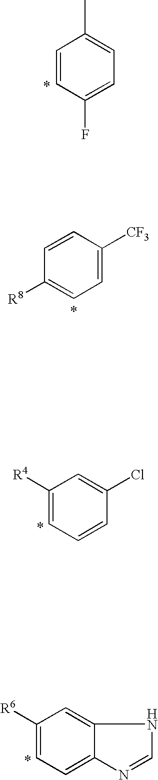
in which R⁸ is —CH₃, fluoro, in which R⁴ is fluoro, in which R⁶ is chloro or —CF₃ chloro, bromo, —CF₃, —CH₃ or chloro —N═C, —CH₃,—OCF₃ or —CH₂OH
R² has the meaning of pyridyl or the group 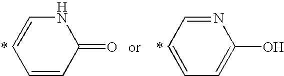
and
R³ has the meaning of hydrogen or fluoro, as well as their isomers and salts.

16. Pharmaceutical compositions according to claim 15 which comprise as compound I and/or II (4-Chlorophenyl)[4-(4-pyridylmethyl)-phthalazin-1-yl]ammonium hydrogen succinate

17. Pharmaceutical compositions according to claims 1-16 which comprise as compound I (4-Chlorophenyl)[4-(4-pyridylmethyl)-phthalazin-1-yl]ammonium hydrogen succinate, sTie2, mAB 4301-42-35, scFv-tTF and/or L19 scFv-tTF conjugate, and as compound II (4-Chlorophenyl)[4-(4-pyridylmethyl)-phthalazin-1-yl]ammonium hydrogen succinatesTie2, mAB 4301-42-35, scFv-tTF and/or L19 scFv-tTF conjugate, with the provisio that compound I is not identically to compound II.

18. Pharmaceutical compositions according to claims 1-17 which comprise as compound I (4-Chlorophenyl)[4-(4-pyridylmethyl)-phthalazin-1-yl]ammonium hydrogen succinate and as compound II sTie2, mAB 4301-42-35, scFv-tTF and/or L19 scFv-tTF conjugate.

19. Pharmaceutical compositions according to claims 1-17 which comprise as compound I mAB 4301-42-35 and as compound II sTie2, and/or scFv-tTF conjugate.

20. Pharmaceutical compositions according to claims 1-17 which comprise as compound I scFv-tTF conjugate and as compound II sTie2 and/or mAB 4301-42-35.

21. Pharmaceutical compositions according to claims 1-17 which comprise as compound I L19 scFv-tTF conjugate and as compound II sTie2.

22. Use of pharmaceutical compositions according to claims 1-21, for the production of a medicament for the treatment of tumors, cancers, psoriasis, arthritis, such as rheumatoide arthritis, hemangioma, angiofribroma, eye diseases, such as diabetic retinopathy, neovascular glaukoma, kidney diseases, such as glomerulonephritis, diabetic nephropathie, maligneous nephrosclerosis, thrombic microangiopatic syndrome, transplantation rejections and glomerulopathy, fibrotic diseases, such as cirrhotic liver, mesangial cell proliferative diseases, artheriosclerosis, damage of nerve tissues, suppression of the ascites formation in patients and suppression of VEGF oedemas.
